(19) United States
(12) Patent Application Publication
(10) Pub. No.: US 2003/0202977 A1
(43) Pub. Date: Oct. 30, 2003

(54) Treatment of osteoarthritis

(75) Inventors: Ashok R. Amin, Union, NJ (US); Steven Abramson, Rye, NY (US); Mukandan Attur, Woodside, NY (US)

Correspondence Address: BROWDY AND NEIMARK, P.L.L.C., 624 NINTH STREET, NW, SUITE 300, WASHINGTON, DC, 20001-5303, US

(73) Assignee: New York University, New York, NY

(21) Appl. No.: 10/461,423

(22) Filed: Jun. 16, 2003

Related U.S. Application Data
(63) Continuation of application No. 09/441,217, filed on Nov. 16, 1999, now abandoned.
(60) Provisional application No. 60/108,521, filed on Nov. 16, 1998.
(60) Provisional application No. 60/116,966, filed on Jan. 22, 1999.

(51) Int. Cl. A61K 39/395

(52) U.S. Cl. 424/146.1; 424/144.1

(57) ABSTRACT

Agents with integrin-afffecting activity, including antibodies and molecules having the antigen-binding portion of such antibodies, are used to regulate inflammatory mediators, including TL-1β, IL-6, IL-8, nitric oxide, PGE₂ and MMPs.

### CROSS-REFERENCE TO RELATED APPLICATIONS

The present application claims priority from U.S. Ser. No. 09/441,217, filed Nov. 16, 1999, which claims priority from provisional applications Serial No. 60/108,521, filed Nov. 16, 1998, and Serial No. 60/116,966, filed Jan. 22, 1999, the entire contents of which are hereby incorporated by reference.

### FIELD OF THE INVENTION

The present invention is directed to a method and composition for regulating inflammatory mediators and, more particularly, for regulating inflammatory mediators in human osteoarthritis-affected cartilage and bovine chondrocytes.

### BACKGROUND OF THE INVENTION

Articular cartilage is avascular, aneural, and alymphatic. Human osteoarthritis-affected cartilage in ex vivo conditions spontaneously releases inflammatory mediators, including nitric oxide (Amin et al, 1995), COS-2 mediated PGE₂ (Amin et al, 1997), IL-6, IL-8 (Dattur et al, 1998), MCP and MMPs sufficient to impair normal cartilage function which leads to cartilage damage.

Chondrocytes hold one of the key positions in the development of arthritis, as it produces the components of the matrix of articular cartilage. The current hypothesis suggests that there may be specific induction and repression of chondrocyte gene expression, controlled through cellular contacts, with the extracellular matrix components in the pericellular environment surrounding the chondrocytes and a complex array of integrins on chondrocytes which may control cartilage homeostasis.

Integrins are heterodimer receptors that contain α and β subunits and bind to extracellular matrix components or membrane bound counter-receptors on other cells (Aplin et al, 1998). Integrins initiate signals that control many cell functions, generally referred to as cell adhesion events. Ligands for integrins are often large extracellular matrix proteins, such as collagen, laminin, vitronectin, osteopontin, and fibronectin. However, although many integrins and the ligands that bind an integrin are described in the literature, the biological function of many of the integrins remains elusive. The integrin receptors comprise a family of proteins with shared structural characteristics of non-covalent heterodimeric glycoprotein complexes formed of α and β subunits. Recently, investigators have reported methods for interacting with some integrins to inhibit adverse consequences in the body, including angiongenesis and inflammation.

Adding monoclonal antibodies that activate α₅β₁ integrins (JBS5) in human osteoarthritis-affected cartilage explants (Shen et al, 1991) significantly augments the release of nitric oxide, PGE₂ and MMP-1 and 9. JBS5 monoclonal antibodies also induced MMP-1 and 9 activity in normal human cartilage. The soluble IL-1 type II receptor (added as a protein or transfected as a cDNA for gene therapy) is a potent inhibitor of nitric oxide, MMPs and PGE₂ production in human cartilage and chondrocytes. The up-regulation of nitric oxide, with or without anti-α₅β₁ monoclonal antibody, could be inhibited by soluble type II IL-1β receptors.

Weerasinghe et al (1998) reported that antibodies against integrin-associated protein could partially block αᵥβ₃-dependent transmigration of monocytes through inflammatory endothelium.

Zheng et al (1998) reported that blocking αᵥβ₃ integrin inhibits cellular migration response to insulin-like growth factor.

Eliceiri et al (1998) and Brooks et al, in U.S. Pat. No. 5,753,230, reported inhibiting angiogenesis by using αᵥβ₃ antagonists.

Yonezawa et al (1996) reported that very late activation antigens, β1 integrins, play an important role with fibronectin in regulating inflammatory cytokine production by human articular chondrocytes.

Duong et al (1998) disclose that PYK2 is highly expressed in osteoclasts and that tyrosine phosphorylation of PYK2 is mediated by β₃ integrins.

Trusolino et al (1998) disclose a growth-factor driven integrin activation mechanism in normal epithelial cells.

Osteopontin is a soluble secretory phosphoprotein with diverse functions which include: marker for commitment to encochondrial ossification (McKee, Glimcher et al, 1992) and inhibition of endotoxin induced nitric oxide production in epithelial cells (Hwang, Lopez et al, 1994; Denhardt, Lopez et al, 1995; Denhardt & Guo, 1993). Osteopontin is secreted by various cell types including activated T cells, macrophages, osteoblasts and hypertrophic chondrocytes. Osteopontin has also been reported to bind to various integrins, including αᵥβ₃ (Denhardt & Noda, 1998). Osteopontin mRNA is up-regulated in human osteoarthritis-affected cartilage as compared to normal cartilage. Addition of osteopontin to human osteoarthritis-affected cartilage inhibits nitric oxide production, whereas antibodies that neutralize osteopontin activity augment nitric oxide production.

Storgard et al (1999) have shown that peptide antagonists to αᵥβ₃ can mimic some of the actions of LM609.

While there have been a number of reports of regulating integrins to inhibit angiogenesis, there have been no reports of interfering with production of specific inflammatory mediators to treat osteoarthritis-affected cartilage and chondrocytes, or of treating autoimmune diseases and diseases involving inflammation.

### SUMMARY OF THE INVENTION

It is an object of the present invention to overcome the aforesaid deficiencies in the prior art.

It is another object of the present invention to provide a method for regulating inflammatory mediators.

It is still another object of the present invention to inhibit the functions of integrins to block inflammatory responses.

It is a further object of the present invention to regulate inflammatory mediators such as IL-1β, IL-6, IL-8, nitric oxide, and PGE₂.

It is yet another object of the present invention to provide an assay to identify anti-inflammatory compounds and/or mediators.

It is a further object of the present invention to provide a screening system to confirm the action of drugs on inflammatory mediators.

According to the present invention, agents which act as either agonists or antagonists to integrins, including antibodies and molecules having the antigen-binding portion of such antibodies, are used to regulate inflammatory mediators, such as nitric oxide, PGE₂ and MMPs.

More specifically, an antibody LM609 (Cheresh, 1987) that inhibits the functions of αᵥβ₃ integrin was found to significantly inhibit either spontaneously released or α₅β₁-induced nitric oxide and PGE₂ production in human osteoarthritis-affected cartilage. This antibody exhibited a dominant negative effect of αᵥβ₃ on α₅β₁ receptor function. Additionally, antibodies to α₅β₁ also up-regulate the accumulation of IL-1β, IL-6, IL-8, together with nitric oxide and PGE₂. That is, the αᵥβ₃ receptor acts as a dominant negative receptor which inhibits the functions of other pro-inflammatory receptors and mediation with respect to induction of inflammatory mediators. α₅β₁ activating antibodies also induced nitric oxide and PGE₂ in primary bovine chondrocytes, which could be inhibited significantly by type II soluble IL-1β receptors, thus mimicking its functions observed in organ cultures of cartilage. The αᵥβ₃ antagonizing antibody also inhibits LPS, IL-1β, IL-1, TNFα and LPS induced inflammatory mediators, which include nitric oxide, in primary bovine chondrocytes. LM609 may block at least one key signal transduction molecule which regulates LPS, IL-l and TNFα induced gene expression. The αᵥβ₃ integrin regulates a broad spectrum of inflammatory mediators that can be induced by LPS, IL-β, TNFα, alone or in combination, irrespective of the angiogenic properties of the diseases.

Furthermore, bovine chondrocytes stimulated with LPS, IL-1β, IL-1+LPS+TNFα and which produce nitric oxide can be inhibited by αᵥβ₃ antibodies (LM609). Rabbit synovial cells stimulated with LPS and IL-1β release PGE₂, which can be inhibited by αᵥβ₃ antibodies. Human endothelial cells stimulated with α₅β₁, or IL-1β induce PGE₂, which can be inhibited by monoclonal antibody to αᵥβ₃ the dominant-negative effect of LM609 was observed in three different cell types.

The antibodies and other molecules that inhibit the function of αᵥβ₃ integrin and related integrins block inflammatory responses, such as those induced in osteoarthritis-affected cartilage in vivo, or in vitro in the presence of LPS, IL-1β, IL-1, TNFα and LPS. The inhibition of activation by these integrins may potentially be mediated by autocrine IL-1 and its receptors. Additionally, the αᵥβ₃ integrin exhibits a negative trans dominant effect on other inflammatory receptors, which include α₅β₁ and IL-1R. Inhibition of the α₅β₁ receptor functions (with αᵥβ₃ monoclonal antibody) also inhibits production of inflammatory mediators.

Previous studies have shown that αᵥβ₃ binds to MMP-2 to promote angiogenesis which can be inhibited by LM609, thus demonstrating the antagonist properties of this antibody (Brooks et al, 1998). LM609 can, thus, work as either an agonist or an antagonist for integrins because:

(a) osteopontin binds to αᵥβ₃ receptor and inhibits the production of inflammatory mediators;

(b) LM609 inhibits the binding of osteopontin, vitronectin, fibrinogen and von Willebrand factor to cells (Cheresh et al, 1987) and, therefore, acts as an antagonist with respect to binding of the ligand to the receptor;

(c) however, LM609 mimics (but does not inhibit) the action of osteopontin with respect to regulation of inflammatory mediators and, therefore, acts as an agonist.

Taken together, these observations suggest that LM609 acts as an antagonist with respect to its ability to inhibit angiogenesis and ligand/receptor binding, but acts as an antagonist and agonist when it comes to regulating inflammatory mediators. Fibronectin and fibronectin fragments which bind to α₅β₁ have been reported to induce IL-6, IL-1 and MMPs (Homandberg et al, 1992, 1997; Yonezawa et al, 1996). The monoclonal antibody JBS5 inhibits the binding of fibronectin to integrins in various cells, but it also induces the production of IL-1, IL-6, IL-8, NO, PGE₂, and MMPs. Thus, it is an activating antibody and acts as an antagonist for ligand binding and an agonist for modulating inflammatory responses. For the purposes of the present invention, then, the compounds of interest will be referred to as agents which affect integrin binding.

It should be noted that another antibody, M13 (Mould et al, 1997), that also binds to α₅β₁ has no effect on nitric oxide but also inhibits the effects of JBS5. Thus, these results suggest that there are activating or inhibiting epitope(s) on the receptor. These activating and inhibiting epitope(s) are also present on αᵥβ₃ receptors because other ligands (CYR61 and Ecstatin), which also bind to the αᵥβ₃ integrin, do not exhibit similar actions as seen with osteopontin and LM609. Therefore, the sequence of the αᵥβ₃ epitope to which LM609 binds is important in sending a dominant negative signal through-the receptor.

Additionally, the sequence of osteopontin that binds to the αᵥβ₃ integrin is important. It should be noted that osteopontin can bind to DC44 and other integrins (Weber et al, 1996), but the sequence of the osteopontin that binds to αᵥβ₃ is important. Obviously, one of these is the RGD sequence in the osteopontin. However, it should be noted that binding of fibronectin to α₅β₁ required RGD and CCBD sequences on fibronectin (Mould et al, 1997).

The LM609 epitope is present in both normal and up-regulated α₅β₁. α₅β₁ can bind to various ligands, including β prothrombin, osteopontin, fibrinogen, CYR61, vitronectin, von Willebrand factor, thrombospondin (reviewed Aplin et al, 1998). The ligand selection by αᵥβ₃ may be controlled by the activation state of this integrin (Byzova et al, 1998). Since LM609 binds to unstimulated bovine chondrocytes and also to activated OA-affected chondrocytes, this epitope on the αᵥβ₃ is exposed in both inactive and activated states of the receptor. This particular epitope is important because it is responsible for triggering the important dominant negative effect.

Other integrins can also induce dominant negative effects on other receptors. Integrins have the property to show trans dominant effects on other receptors, e.g.:

(a) αᵥβ₃ integrin-deficient neonatal mice show trans dominant inhibitor effects on other integrins with respect to cytoskeletal assembly (Hodivala-Dilke et al, 1998); and

(b) similarly, β1 integrin cytoplasmic subdomains show dominant negative functions-with respect to formation of focal adhesion complex (Retta et al, 1998).

### BRIEF DESCRIPTION OF THE DRAWINGS

FIG. 1a shows regulation of NO and PGE₂ in primary bovine chondrocytes by integrin receptors in the presence of sIL-1RII receptors. Bovine chondrocytes were seeded in 24 well plates at a density of 0.5×10⁶ cells/well in 2 ml medium. After 24 hours post-seeding, the cells were treated with either 50 ng of JBS5 monoclonal antibody or 1 ng/ml IL-1β alone and in the presence and absence of 0.5 μg/ml of sIL-1RII for 72 hours. NO and PGE₂ were estimated as described in Materials and Methods. The data present one of the two similar experiments. Data represent mean±s.d. value. The p values described are compared with IL-l (*) or anti-α₅β₁ monoclonal antibody (**); *p≦0.01, **p≦0.01.

FIG. 1b shows regulation of NO and PGE₂ in osteoarthritis-affected cartilage by integrin receptors in the presence of sIL-1RII. Human osteoarthritis-affected cartilage organ cultures were set up as described in Materials and Methods. The cultures were treated with anti-α₅β₁ monoclonal antibody or IL-1β alone in the presence and absence of sIL-1RII as described above. NO and PGE₂ were estimated at the end of the experiment. The data represent the mean±s.d. values. The p values described are compared with spontaneously released (*), IL-1β induced (**) or anti-α₅β₁ monoclonal antibody induced (#) NO and PGE₂.

FIG. 2 shows regulation of IL-1β mRNA by integrins of bovine chondrocytes. Normal bovine chondrocytes were incubated with various modulators for 72 hours and analyzed for IL-1β mRNA and GAPDH in the same experiment by RT-PCR as described in Materials and Methods. The 350 bp IL-1β and 200 bp GAPDH fragments were sequenced to ascertain their identity. Lane 1 shows uninduced cells; lane 2, 100 μg/ml LPS; lane 3, anti-αᵥβ₃ monoclonal antibody+LPS; lane 4, anti-α₅β₁ monoclonal antibody; and lane 5, anti-αᵥβ₃ monoclonal antibody and anti-α₅β₁. The αᵥβ₃ monoclonal antibodies were preincubated for fifteen minutes before inducing the cells with various modulators. The data represent one of the two similar experiments.

FIG. 3a shows regulation of NO and PGE₂ by αᵥβ₃ integrin in bovine chondrocytes. Bovine chondrocytes were seeded in 24 well plates and stimulated with 1 ng/ml Il-1β, 100 μg/ml LPS, IL-1β+LPS+TNFα and anti-α₅β₁ monoclonal antibody in the presence or absence of 5 μg/ml anti-αᵥβ₃. The accumulation of NO and PGE₂ was estimated at the end of 72 hours. The data represent one of the two similar experiments the data represent mean±s.d. value.

FIG. 3b shows regulation of NO and PGE₂ by αᵥβ₃ integrin in human osteoarthritis-affected cartilage. Human osteoarthritis-affected cultures were incubated in organ cultures as described in Materials and Methods. The cultures were preincubated in with 5 μg/ml of anti-α₅β₁ monoclonal antibody and stimulated with 1 ng/ml IL-1β or IL=1β+TNFα+LPS, or anti-α₅β₁ monoclonal antibody, and the levels of NO in μM/g and PGE₂ in ng/ml/g were estimated at the end of 72 hours. The data represent one of two similar experiments. The data represent mean±s.d. value.

FIG. 4a shows regulation of PGE₂ by anti-αᵥ-12-β₃ in rabbit synovial cells and FIG. 4b shows regulation of PGE₂ by anti-αᵥβ₃ in rabbit endothelial cells. Rabbit synovial cells and HUVEC cells were grown as described in Materials and Methods. The cells were situated with LPS, IL-1β, or anti-α₅β₁ monoclonal antibody after preincubation with anti-LM609. The data represent one of two similar experiments. The data represent mean±s.d. value.

FIG. 5 shows regulation of inflammatory mediators by integrins in cartilage. These data show the hypothetical scheme on the mechanisms of action of anti-α₅β₁ and anti-αᵥβ₃ monoclonal antibody in cartilage and chondrocytes. The anti-α₅β₁ or αᵥβ₃ monoclonal antibody induced positive or negative signals to up-regulate IL-1β gene expression in cartilage. This may also happen due to the increase in thr production of fibronectin and osteopontin in human osteoarthritis-affected cartilage. The up-regulated IL-1β mRNA induces IL-1β production in an autocrine fashion which also up-regulates other inflammatory mediators such as NO, PGE₂, IL-6 and IL-8.

### DETAILED DESCRIPTION OF THE INVENTION

According to the present invention, inflammatory mediators, including IL-1β, IL-6, IL-8, nitric oxide, PGE₂, and MMPs, are regulated by administering to a patient in need thereof an effective amount of antibodies that inhibit the function of at least one integrin. The preferred integrin to be inhibited is αᵥβ₃ although antibodies to αᵥβ₅ may also be administered to inhibit inflammatory responses. This inhibition of inflammatory responses is independent of any angiogenic properties of the tissue affected. More specifically, inflammation can be mediated by administering LM609, a mutein, fragment, or analog thereof, or a compound that mimics the activity of LM609. For purposes of the present invention, unless stated to the contrary, a reference to LM609 includes LM609 per se, as well as analogs, muteins, fragments, and analogs thereof.

The human IL-1 type II decoy receptor (IL-1RII) inhibits production of IL-1, nitric oxide, PGE₂, IL-6, IL-8 and MMPs in human arthritis affected cartilage in ex vivo conditions in the presence or absence of IL-1.

Cells (chondrocytes or synovial cells) transfected with the IL-1RII cDNA in an expression vector produce a functional receptor which inhibits IL-1 mediated functions which include IL-1, nitric oxide, PGE₂, IL-6, IL-8 and MMPs in human arthritis affected cartilage in ex vivo conditions.

Where inflammatory mediators contribute to the cause of or to the pathology associated with a disease or condition, regulation of these inflammatory mediators reduces the deleterious effects of the inflammatory mediators. By inhibiting inflammatory mediators, one can intervene in the disease, ameliorate the symptoms and, in some cases, cure the disease.

The method of the present invention is particularly effective because the therapy is highly selective for inflammatory mediators and not for other biological processes. The discovery that antibodies to specific integrins can regulate inflammatory mediators allows for the development of therapeutic compositions with potentially high specificity and, therefore, relatively low toxicity. Thus, although the invention discloses the use of monoclonal antibodies which may have the ability to inhibit one or more integrins, one can design compositions which selectively inhibit the integrin of interest and, therefore, do not have the side effect of inhibiting other biological processes other than those mediated by the integrin of interest.

The action of the LM609 specific epitope on the αᵥβ₃ integrin and the osteopontin specific epitope is important because:

(a) CYR61, another ligand that also binds to αᵥβ₃ promotes angiogenesis and tumor growth, whereas LM609 inhibits angiogenesis and tumor growth (Brooks et al, 1994; Babic et al, 1998).

(b) The toxin (Ecstatin) which also binds to α₅β₁ preferentially inhibits PGE₂ production more than nitric oxide production in chondrocytes, whereas LM609 and osteopontin equally inhibit nitric oxide and PGE₂.

(c) LM609 mimics the functions of osteopontin that inhibit nitric oxide and PGE₂ production. Thus, the antibody acts as an agonist with respect to the action of osteopontin on αᵥβ₃ integrins.

It should be noted that osteopontin is a multi-functional protein, and this property of anti-inflammatory activity may be confirmed to a particular amino acid sequence or structure of osteopontin, cf. Young et al (1990).

Thus, the target(s), including αᵥβ₃ of the antibody LM609 can be used to treat a variety of diseases or conditions in which IL-1β, nitric oxide, PGE₂, IL-6 and IL-8 play a role in the disease process and medical conditions. Additionally, the trans dominant negative effect of LM609 broadens the scope of this reagent.

Extracellular matrices regulate inflammatory mediators in normal and osteoarthritis-affected cartilage, and primary bovine chondrocytes were studied. It was found that:

(1) Antagonist antibody to αᵥβ₃, that inhibits the binding of fibronectin, also acts as an agonist by inducing NO, PGE₂, IL-6, IL-8 production in an autocrine IL-1β dependent fashion.

(2) Similarly, antagonist antibodies to αᵥβ₃ that inhibit the binding of osteopontin, act as an agonist by inhibiting the spontaneous production of NO, PGE₂, IL-6 and IL-8 production induced through autocrine IL-1β.

(3) Furthermore, the function blocking anti-αᵥβ₃ antibody (LM609) inhibits the function of anti-αᵥβ₃ monoclonal antibody, IL-1β and LPS with respect to induction of NO, PGE₂, and IL-1β production in a dominant negative.

### Materials and Methods

Reagents

ELISA kits for IL-1β, IL-6 and IL-8 were purchased from R and D (Minneapolis, Minn.). Cyclic RGD and RGE peptides were procured form Bachem Biosciences, Inc. (King of Prussia, Pa.). Anti-αᵥβ₃ (JBS5) and anti-3 (LM609) were purchased from Chemicon International (Temecula, Calif.). Another anti-αᵥβ₃ antibody (mAb13) was a generous gift from Dr. Yamada (NIH, Bethesda, Md.).

Procurement of Human Cartilage

Cartilage slices were taken from the knees of patients who had been diagnosed with advanced osteoarthritis, ages 50-70 years, who were undergoing knee replacement surgery. Non-arthritic knee cartilage, normal control, ages 20-70 years, was obtained from patients with fractures or from accident victims after knee amputation.

Osteoarthritis-Explant Assay

Knee articular cartilage from osteoarthritis patients or normal individuals was cut into 3 mm discs, and 4-6 discs were placed (triplicate or quadruplicate) into a 24-well plate containing 2 ml F-12 medium+0.1% human albumin in the presence or absence of anti-αᵥβ₃ (JBS5) or anti-3 (LM609) antibodies along with various modulators, as previously described by Amin et al (1995 and 1997).

Isolation of Bovine Chondrocytes

Bovine chondrocytes were isolated as previous described by Attur et al 1998. Briefly, cartilage slices from bovine cartilage were finely minced and digested sequentially with 0.2% testicular hyaluronidase for five minutes at 37° C., and 0.2% collagenase for 1-2 hours at 37° C. After being strained through sterile nylon mesh, the cells were centrifuged at 1200×g for ten minutes. The cell pellets were washed twice with Ham's F-12 medium, followed by resuspension and centrifuging at 1000×g for ten minutes. Finally, the pellets were suspended in complete F-12 medium before being plated to flasks.

Cultivation of HUVEC Cells

Human umbilical vein endothelial cells (HUVEC) were passaged in medium M199 (Gibco BRL, MD) containing 20% fetal bovine serum, 4 mM L-glutamine, penicillin, streptomycin, and 2 ng/ml bFGF on 0.2% gelatin (Sigma Chemical Company, St. Louis, Mo.) coated 80 mm diameter dishes. HUVECs were not used after the sixth passage. For experimental purposes, HUVECs were plated onto 0.2% gelatin coated dishes and allowed to form a monolayer. The cultures were serum starved with unsupplemented medium M199 containing 0.5% fetal bovine serum for 16-18 hours.

Cultivation of Rabbit Synovial Cells

Rabbit synovial cells (HIG82), Georgescu et al (1998), were kindly provided by Dr. Michael Pillinger, Dept. of Rheumatology, Hospital for Joint Diseases, and grown in Ham's F-12 medium containing 10 fetal bovine serum, 4 mM L-glutamine, penicillin and streptomycin. Before the experiment the cells were starved with Ham's F-12 medium containing 0.5% fetal bovine serum overnight for 14-16 hours. The medium was changed and the cells were preincubated with 50 μg/ml anti-3 antibodies (LM609) for 30 minutes before being stimulated with 5 ng/ml IL-1β or 100 μg/ml LPS. PGE₂ was measured 72 hours post-stimulation.

FACS analysis was performed using rabbit synovial cells, and bovine chondrocytes were stained with LM609 and a secondary PE labeled antibody.

Analysis of Protease Activity in Gels

Matrix metalloproteases (MMPs) activity was determined using zymogram gels (Novex, San Diego, Calif.). Briefly, the enzyme sample was denatured in DSD without reducing agent and run on zymogram gel containing 1% gelatin or casein using Tris-glycine SDS running buffer. The enzyme was then renatured by soaking the gel in Novex renaturing buffer. The gel was equilibrated in Novex developing buffer and then stained in Coomassie blue to visualize a clear band at the site of the digested substrate. The quantitation was performed by densitometry. The metalloproteases were identified based on Western blot analysis of duplicate gels using the antibodies from the above source.

Nitrate and PGE₂ Analysis

NO production was measured by estimating the stable NO metabolite, nitrite, in condition medium by modified Griess reaction, Gilliam et al (1993). PGE2 was analyzed from supernatant by RIA, Amin et al (1997).

RT-PCR Analysis of IL-1β

One microgram of total RNA isolated from bovine chondrocytes from various experiments was used for cDNA synthesis (GIBCO BRL) and PCR analysis. The sense primer 5′-GCGCCTGGTCACCCAGGGCTGC-3′ (SEQ ID NO:1) and antisense primer 5′-GGATCCTCCCCCGCTCCTGGAAGATC-3′ (SEQ ID NO:2) were used for amplifying GAPDH (DiCesare et al, 1998). RT-PCR analysis of IL-1β was performed using the sense primer 5′-GAAGAGCTGCATCCAACACC-3′ (SEQ ID NO:3) and antisense primer 5′-ATGCAGAACACCCACCTTCTCG-3 (SEQ ID NO:4).

Full-length soluble type II IL-1 receptor was cloned from human neutrophils using RT-PCR. The forward primer 5′-CGGGATCCATGTTGCGCTTGTACGTGT-3′ (SEQ ID NO:5) introduced a BamHI site at the 5′ end of the ATG and reverse primer 5′-TAAAGCGGCCGCTCACTTGGGATAGAATTG-3′ (SEQ ID NO:6) introduced a NotI site immediately following the stop codon. The PCR amplified DNA was digested with BamHI and NotI and the fragment, 1196 base pairs, was sub-cloned into a pFAST BAC-1 BACMID. The recombinant sIL-1RTI was generated using the BAC to BAC system as described in Patel et al (1997). The biologically active sIL-1βRII released from Sf9 insect cells was used for inhibition studies.

Statistical Analysis

Data were expressed as mean±S.D. and statistical analysis was performed using GraphPad Software (v. 1.14). The T test or non-parametric (Mann-Whitney or Wiulcoon test) was performed to analyze the data.

### Results

Role of Integrin on Production of Inflammatory Mediators in Human Osteoarthritis-Affected Cartilage

The present inventors recently observed that the autocrine production of IL-1β depends on the levels of intra-acicular osteopontin. Increase in the levels of osteopontin inhibits IL-1β-mediated NO and PGE₂ production in osteoarthritis-affected cartilage, whereas anti-osteopontin anti-serum augments IL-1β-mediated NO production under the same experimental conditions (Attur, 1999). In view of the above observations and previous reports that inflammatory mediators are up-regulated and spontaneously released from osteoarthritis-affected cartilage, the present inventors examined if the induction of these inflammatory mediators in human osteoarthritis-affected cartilage was attributed to stimulation/modulation of integrins. Previous studies have shown that antibodies to various integrins, mainly 3, αᵥβ₃ α₂β₁ α₆β₁ and α₁β₁αᵥβ₃ modulate function, such as cell growth, differentiation, apoptosis, adhesion and angiogenesis (Aplin et al, 1998). In the present study, the use of natural ligands was avoided because of the multi-functional role of fibronectin and osteopontin and their ability to bind various receptors (Aplin et al, 1998; Longhurst et al, 1998; Hocking et al, 1998; Vogel et al, 1997).

Monoclonal antibodies were selected rather than the natural ligands to ascertain the specificity in the following experimental protocols. Furthermore, monoclonal antibodies induced high affinity binding and oligomerization of integrins without changes in integrin gene expression (Aplin et al, 1998; Longhurst et al, 1998). The monoclonal antibodies used were those which have been reported to act as antagonists to inhibit the binding of the αᵥβ₃ or 3 positive cells to plates coated with fibronectin or vitronectin, respectively, but which also act as agonists to mimic the function of their respective ligands as previously reported for the T cell receptor, such as some anti-CD3 antibodies. JBS5 is an anti-αᵥβ₃ monoclonal antibody that inhibits the binding of fibronectin and causes aggregation of the receptors and homotypic clustering in Jurkat T cells (Shen et al, 1991; Kanbey, 1991). LM609 is an anti-3 monoclonal antibody that blocks the binding of its ligands to the receptor and also inhibits functions, such as angiogenesis, in various experimental systems (Brooks et al, 1994).

The effects of anti-αᵥβ₃ (JBS5) and anti-3 (LM609) monoclonal antibodies were evaluated on the regulation of spontaneously released NO, PGE₂, IL-6, IL-8 and IL-1β in human osteoarthritis affected cartilage in ex vivo conditions as described above. Human osteoarthritis-affected cartilage (control) spontaneously released NO, PGE₂, IL-6, IL-8 and IL-1β as previously reported by Amin et al (1995 and 1997) and Patel et al (1998). Addition of JBS5 monoclonal antibodies showed significant augmentation (p≦0.01) of NO, PGE₂, IL-6, IL-8, and IL-1β production as compared to the control cartilage, shown in Table 1. In contrast, the addition of LM609 monoclonal antibody showed inhibition of NO, PGE₂, IL-6, IL-8 and IL-1β productions as compared to the control.

TABLE 1
Regulation of IL-1β, IL-6, IL-8, NO and PGE₂
by Integrin in Human OA-Affected Cartilage

| | per 100 mg Cartilage | | | | |
|---|---|---|---|---|---|
| | Nitrite | PGE₂ | IL-1β | IL-6 | IL-8 |
| Conditions | (μM) | (ng/ml) | (pg/ml) | (ng/ml) | (ng/ml) |
| OA-Affected Cartilage | 1.2 ± 0.2 | 13.1 ± 0.2 | 4.0 ± 1.0 | 7.3 ± 3.0 | 9.3 ± 2.7 |
| (Control) | | | | | |
| + RGD | 3.8 ± 1.0^{b} | ND | 11.1 ± 2.0^{b} | 5.2 ± 4.2^{f} | 5.1 ± 4.6^{f} |
| + RGE | 0.4 ± 0.2^{c} | ND | ND | ND | ND |
| + anti-α₅β₁ | 12.7 ± 2.4^{a} | 19.1 ± 3.2^{c} | 87.6 ± 10.0^{a} | 17.7 ± 4.4^{a} | 17.7 ± 4.0^{b} |
| + anti-αᵥβ₃ | 0.5 ± 0.2^{b} | 8.2 ± 2.2^{b} | 2.5 ± 1.0^{b} | 2.9 ± 1.9^{e} | 5.7 ± 0.8^{b} |
| + NMMA | 0.8 ± 0.1^{c} | 18.4 ± 0.8^{c} | ND | ND | ND |
| + IL-1β | 17.3 ± 2.4^{a} | 29.6 ± 7.2^{c} | ND | ND | ND |

^{a}≦ 0.001
^{b}≦ 0.01
^{c}≦ 0.03
^{d}≦ 0.04
^{e}≦ 0.05
^{f}≦ 0.01
ND = Not Done

OA-affected cartilage was incubated in the medium with various modulators: 0.5 μg/ml of α₅β₁ (JBS5), 5 μg/ml αᵥβ₃ Abs, L-NMMA (500 μM), IL-1β (1 ng/ml), 1 mg/ml of RGD or RGE. The accumulation of IL-1β, IL-8, IL-6, nitrite and PGE₂ were estimated 72 hours post stimulation. Data represents±S.D. where n=3. The P values described are compared with the (control) unstimulated OA-affected cartilage. The data represent one of the two similar experiments.

IL-1β, as expected (positive control) augmented the release of NO and PGE₂ in osteoarthritis-affected cartilage. An additional control also showed that inhibition of NO by a NOS inhibitor (L-NMMA) augmented PGE₂ production as previously reported by Amin et al, 1997. These experiments suggest that modulation of αᵥβ₃ and 3 integrins regulates pleiotropic signaling molecules known to be regulated by IL-1β.

Arner et al (1994), have reported that cyclic peptides with RGD sequence, which are ligand binding motifs identified as recognition sequences in ECM proteins, induce MMPs in chondrocytes. The effects of cyclic RGD and RGE (negative control) were tested on NO production in osteoarthritis-affected cartilage. The RGD peptide showed a significant augmentation of NO accumulation, whereas the RGE peptide showed significant inhibition of NO accumulation as compared to control cartilage. These experiments suggest that, although RGD and CCBD motifs are both required to modulate the αᵥβ₃ integrin by fibronectin, these RGD/RGE peptides partially mimic the action of their ligands and confirm that extracellular matrix-integrin interaction can modulate NO and PGE₂ production in osteoarthritis-affected cartilage, similar to that observed with IL-1β.

These experiments demonstrate that antibodies to αᵥβ₃ and 3 not only block the binding of fibronectin and osteopontin to their respective receptors like an antagonist, as previously reported by Loeser (1993), Moule et al (1996) Cheresh et al (1987), but also act as agonists to their receptors and mimic the action of fibronectin and osteopontin, respectively. These data support the hypothesis that increased accumulation/synthesis of fibronectin and its proteolytic fragments may interact with its prototypic receptor: αᵥβ₃ integrin and induce NO, PGE₂, IL-6, IL-8, and IL-1β production in human osteoarthritis-affected cartilage. Osteopontin may interact with its protypic receptor: 3 integrin and inhibit NO, PGE₂, IL-6, IL-8 and IL-1β production in osteoarthritis-affected cartilage. In view of the inhibitory effect of LM609 monoclonal antibody on NO, PGE₂, IL-6, IL-8 and IL-1β production, it appears that increased osteopontin accumulation in osteoarthritis-affected cartilage may have a protective function by containing the action of IL-1β and maintaining cartilage homeostasis.

It should be noted that osteopontin and LM609 antibody both inhibited NO production. LM609 has been reported to inhibit angiogenesis-and tumor growth (Brooks et al, 1994; Beuerlein et al, 1998), but other ligands, such as CYR6 or Dell of the 3 receptor promote angiogenesis and neovascularization (Babic et al, 1998). This is not surprising because Echistatin, a snake venom protein that also binds to 3 (Zheng et al, 1998) shows differential regulation of inflammatory mediators distinct from those observed with osteopontin. These results suggest that the type (ligand or antibody) and the epitope to which it binds in the binding pocket of 3 is critical for the type of signal relayed by this receptor.

Up-Regulation of NO, PGE₂, IL-6, IL-8 and IL-1β by Anti-αᵥβ₃ Monoclonal Antibody and RGD in Normal Human Cartilage

It has previously been shown that human osteoarthritis-affected cartilage releases various inflammatory cytokines, including IL-1β, that act in an autocrine fashion. The action of this autocrine IL-1β could be neutralized by adding soluble type IL-1β receptors (IL-1RI). Therefore, the effect of anti-αᵥβ₃ and 3 monoclonal antibodies was tested on human cartilage. As observed in osteoarthritis-affected cartilage, normal cartilage showed increased accumulation of IL-1β (p<0.001), and IL-8 (p≦0.001) in the presence of JBS5 monoclonal antibody, whereas monoclonal antibodies to 3 (LM609) and RGE peptide had no significant effects. These experiments suggest that normal human cartilage, like osteoarthritis-affected cartilage, is also equally susceptible to the triggering of the αᵥβ₃ integrin with respect to producing inflammatory mediators.

Autocrine Production of IL-1β is Required for Induction of NO and PGE₂

In view of the above experiments, it was hypothesized that activating αᵥβ₃ integrin may initiate the initial insult of IL-1β in normal chondrocytes or cartilage. NO and PGE₂ were studied. The role of autocrine IL-1β in the regulation of NO and PGE₂ production in primary bovine chondrocytes was tested by stimulating the cells with IL-1β +recombinant soluble type II IL-1 receptors (sIL-1RII) or anti-αᵥβ₃ monoclonal antibody±sIL-1RII. Addition of IL-1β or anti-αᵥβ₃ monoclonal exhibited up-regulation of NO and PGE₂ in primary bovine chondrocytes. Preincubation of cells for fifteen minutes with sIL-1RII not only blocked the IL-1β induced accumulation of NO and PGE₂. but also blocked anti-αᵥβ₃ monoclonal antibody induced production of NO and PGE₂. These experiments suggest that autocrine production of IL-1β is required for induction of NO and PGE₂ by αᵥβ₃ Monoclonal antibody 13, another anti-αᵥβ₃ antibody which inhibits β1 function, was tested on the regulation of inflammatory mediators in bovine chondrocytes. Antibody mAb13, which recognizes an epitope on the receptor which competes with RGD peptide an dCCME fragment of fibronectin, therefore, decreases the binding of fibronectin. This antibody, unlike JBS5 or in the presence of JBS5, had no significant effect on the production of inflammatory mediators in these cells.

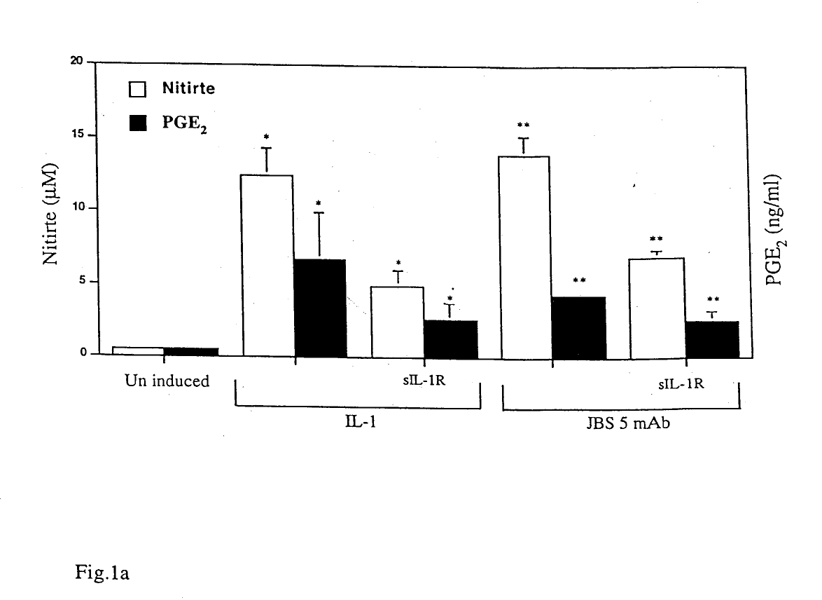

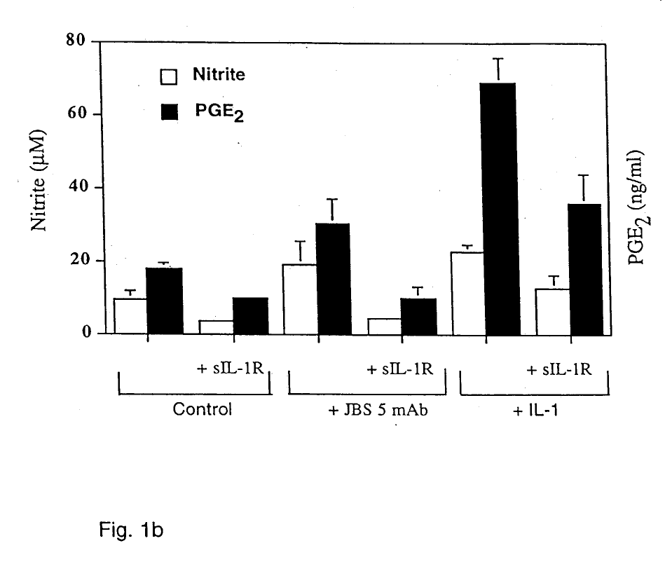

Thus, it appears that the spontaneous production of NO and PGE₂ and the autocrine production of IL-1β in osteoarthritis-affected cartilage may be induced by αᵥβ₃ integrin. Addition of the JBS5 monoclonal antibody of IL-10 augmented the production of NO and PGE₂ in human osteoarthritis-affected cartilage. The recombinant sIL-1RII inhibited significantly (p<0.091) both the IL-1β and JBS5 monoclonal antibody induced NO and PGE₂ production in these osteoarthritis cartilage explants, shown in FIG. 1b.

These experiments on the whole demonstrated that the action of anti-αᵥβ₃ monoclonal antibody, as observed in cartilage organ cultures, could be mimicked in monolayer cultures of primary chondrocytes in vitro. Also, sIL-1RII blocks the effect of the autocrine loop of IL-1β on NO and PGE₂ production induced by JBS5 monoclonal antibody similar to that observed with IL-1β induction. These experiments also suggest that the initial insult of IL-l in normal cartilage may be induced or augmented by integrin receptors during the early stages of osteoarthritis.

Regulation of IL-1β mRNA in Bovine Chondrocytes by Integrins

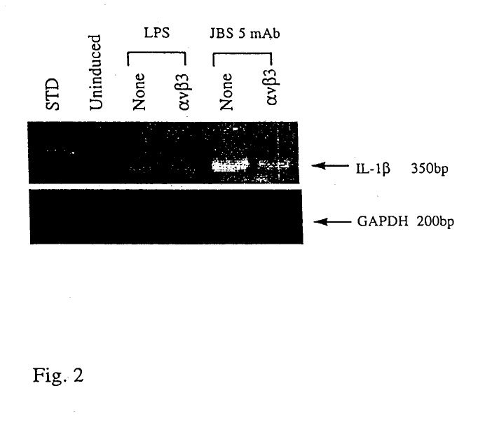

The gene expression of IL-1β in chondrocytes stimulated with anti-αᵥβ₃ monoclonal antibody was examined under various parameters. Normal bovine chondrocytes were stimulated with LPS, anti-3 monoclonal antibody+LPS, JBS5 monoclonal antibody, and LM609+JBS5. LPS and anti-αᵥβ₃ monoclonal antibody showed up-regulation of IL-1β mRNA accumulation as analyzed by RT-PCR analysis, whereas LPS+LM609 monoclonal antibody and LM609+JBS5 monoclonal antibodies inhibited IL-1β mRNA accumulation, as shown in FIG. 2. These experiments further implicate the role of integrins in regulating IL-1β gene expression in chondrocytes together with autocrine production of IL-1β. Previous studies have shown that fibronectin increases IL-1β production, which is sensitive to cycloheximide and actinomycin D (Homandberg et al, 1997). These experiments further strengthen the hypothesis that the fibronectin may be acting via the αᵥβ₃ integrin. These experiments also suggest that the 3 integrin negatively modulates IL-1β expression which, in turn, may influence other inflammatory mediators. It appears that the integrin regulates αᵥβ₃ in a dominant negative fashion.

Anti-3 Integrin Monoclonal Antibodies Inhibit the Action of αᵥβ₃, IL-1β, LPS and IL-1β+TNFα+LPS Induced NO and PGE₂ Production

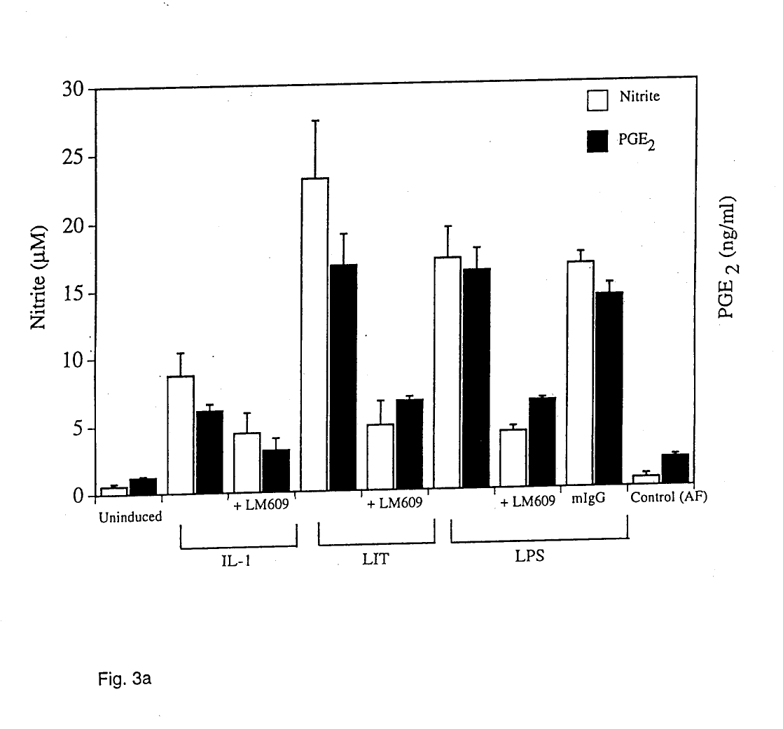

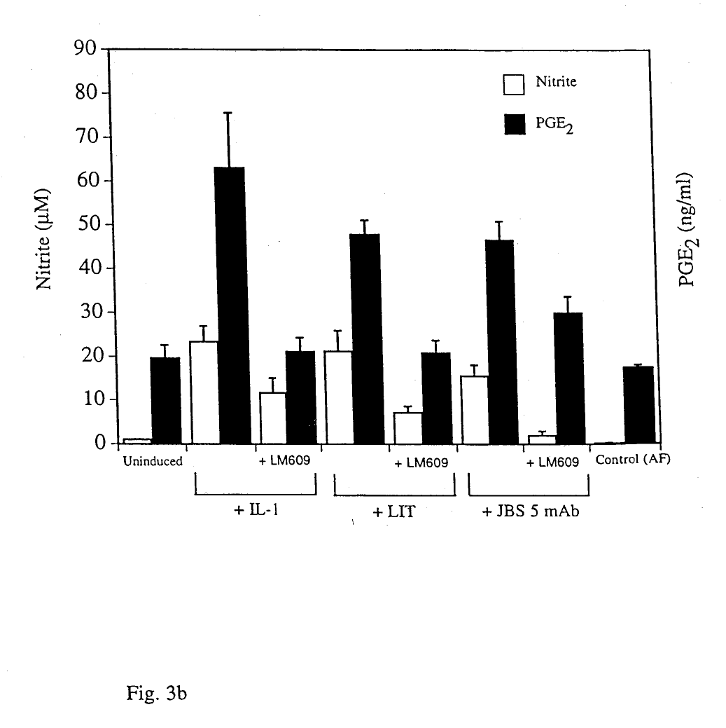

Expression of 3 on bovine chondrocytes and rabbit synovial cells (HIG82) was confirmed by FACS analysis. The role of LM609 integrin monoclonal antibody on the regulation of other pro-inflammatory receptors was studied. Normal bovine chondrocytes were preincubated for fifteen minutes with anti-3 monoclonal antibody and stimulated with anti-α₅β₃ monoclonal antibody, IL-1β, LPS and IL-1β+TNFα+LPS. The levels of NO and PGE₂ were estimated. It was found that the anti-3 monoclonal antibody inhibited, in a trans dominant fashion, induction of NO and PGE₂ by anti-αᵥβ₃ monoclonal antibody, IL-1β, LPS, or a combination of IL-1β+TNFα+LPS, as shown in FIG. 3a. Similar results were also observed in osteoarthritis-affected cartilage that were induced with anti-αᵥβ₃ monoclonal antibody, IL-1β, and IL-1β with TNFα and LPS, as shown in FIG. 3b.

In view of the above observations, the effect of LM609 on PGE₂ production was tested in two other cells.

Regulation of PGE₂ in Rabbit Synovial Cells by Anti-3 Monoclonal Antibody

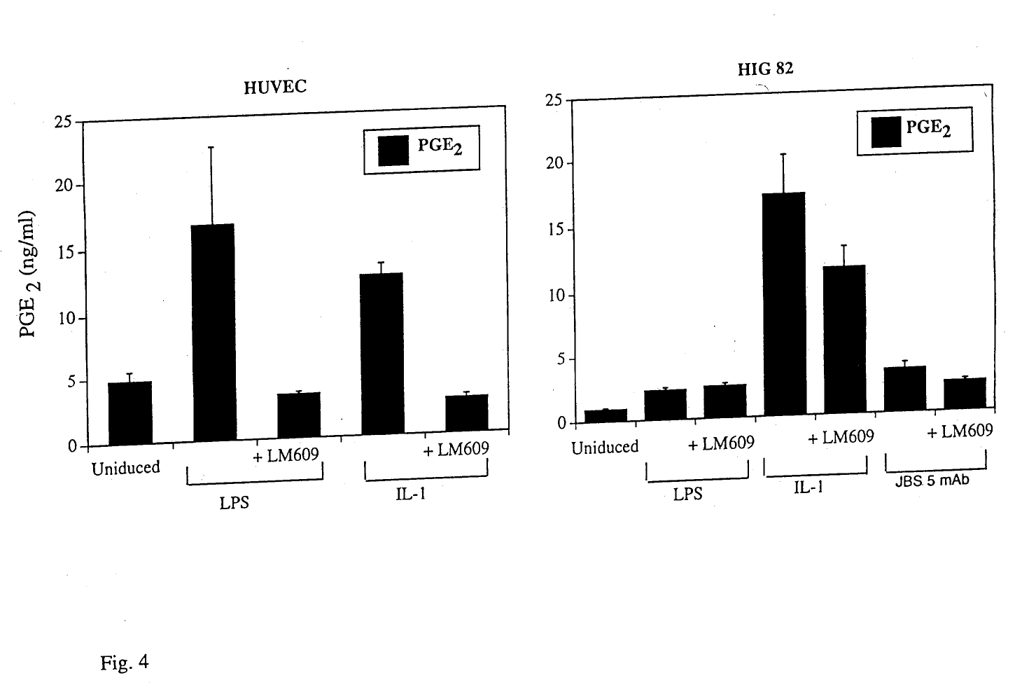

Rabbit synovial cells (HIG82) were found to increase PGE₂ production in response to IL-1 stimulation (Georgescu et al, 1988). These cells were stimulated with LPS and IL-1β to induce PGE₂ in the presence or absence of LM609. Fifteen minutes of preincubation with LM609 significantly inhibited the production of PGE₂ by rabbit synovial cells induced by IL-1β and LPS, as shown in FIG. 4a. Storgard et al (1999) reported that LM609 inhibited angiogenesis of synovial lining in the joints of arthritis model rabbis (Storgard et al, 1999). Recently, the role of cyclooxygenase-2 in the regulation of angiongenesis has been identified (Tsujii et al, 1998). It is important to know that the inhibitory effect of LM609 in this model may be due to its inhibitory effect on cyclooxygenase expression. These experiments as observed with chondrocytes showed inhibition of PGE₂ production by anti-3 monoclonal antibodies in a trans dominant fashion in cells stimulated with LPS and IL-1β.

Regulation of PGE2 in Endothelial Cells by Anti-3 Integrin

The effect of LM609 was tested on the production of PGE₂ in primary human umbilical vein endothelial cells HUVEC) that were stimulated with LPS, IL-1β, or JBS5 monoclonal antibody. As expected, the monoclonal antibody LM609 had no significant effect on the production of PGE₂ in these cells. This is due to low or no 3 receptors on normal HUVEC (Eliceiri et al, 1998).

These experiments demonstrate trans dominant inhibition of αᵥβ₃, IL-1β, LPS and IL-1β +TNFα+LPS induced functions by 3, suggesting a “cross-talk” between αᵥβ₃, IL-1β, LPS, and TNFα receptors and 3 receptors in human and bovine chondrocytes and rabbit synovial cells. The possibility of 3 co-associating the IL-1β cannot be resulted out, as 3 receptors have been reported to associated with CD47 in regulating pro-inflammatory cytosine in human monocyte (Hermann et al, 1999). The association of 3 with PDGFβ, IRS-1 as well as the dependence of 3 for biological effect of FGF-2 indicate that 3 is linked with the cellular responses of certain growth factors (Vuori et al, 1994). It should be noted that most of the experiments were performed in ex vivo conditions without exogenous growth factors, as dissection of integrin signaling pathways may be further complicated by the cellular response to growth factors, such as IGF and EGF, which can mediate similar intracellular responses as integrin-ligand interactions (Kumar, 1998).

Previous studies have shown that expression of αᵥβ₃ in K562 cells inhibits phagocytic functions of the endogenous α₅β₁ (Blystone et al, 1994). Similarly, muscle-specific common receptor α₇β₁ negatively regulates α₅β₁ receptor function (Tomatis et al, 1999). This intracellular trans dominant inhibition of 3 may be due to a blockade of integrin signaling and/or conformational changes in the extracellular domain induced by the β₃ cytoplasmic tail of the suppressive integrin as observed with αIIbβ₃ integrin (Diaz-Gonzalez et al, 1996), or inhibition of intracellular signaling molecules induced by αᵥβ₃, IL-1β, LPS, and TNFα receptors. This assumption is supported by the recent observations that suggests that:

(a) p16^{INK4A} tumor suppressor protein inhibits 3 integrin-mediated cell spreading on vitronectin coated plates by blocking PKC-dependent localization of 3 to focal contacts (Hraeus et al, 1999)

(b) Tumor suppressor PTEN inhibits fibronectin-binding integrin and growth factor-mediated mitogen activated protein (MAP) kinase ignaling pathway (Gu et al, 1998).

(c) Ephrin B-1 induced increase in cell attachment is involved in “inside out” activation of α₅β₁ integrin in HEK293 and αᵥβ₃ integrin in endothelial cells (Huynh-Do et al, 1999).

Up-regulation of MMP-1 and -9 activity in human osteoarthritis-affected cartilage exposed to anti-αᵥβ₃ monoclonal antibody has been observed, based on increased activity in gelatin impregnated gels. This up-regulation can be inhibited by 3. These experiments were similar to those reported in rheumatoid synovial fibroblasts producing collagenase which could be inhibited by anti-3 monoclonal antibody (Sarkissian et al, 1999). αᵥβ₃ receptors are also reported to be associated with other proteases such as urokinase metalloprotease 2A and its receptor UPAR (Gladson et al, 1995).

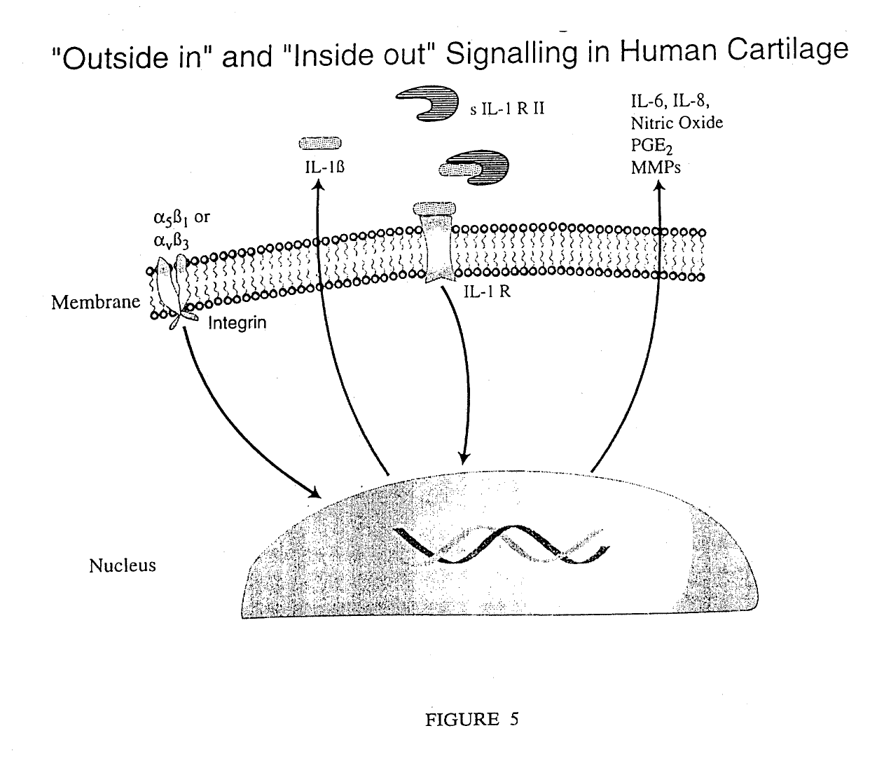

The experiments described above show two well-characterized monoclonal antibodies that specifically bind to α₅β₁ and αᵥβ₃ integrin and mimic the action of their ligands (fibronectin and osteopontin) like receptor agonist (cf. FIG. 5). These studies suggest that antibodies to αᵥβ₃, induce or augment IL-1β production which, in turn, is responsible for induction of NO, PGE₂, IL-6, IL-8 and MMPs, whereas αᵥβ₃ which is over expressed in the superficial zone of osteoarthritis-affected cartilage as compared to normal cartilage (Ostergaard et al, 1998), down-regulates IL-1β expression and the effects of IL-1β in a trans dominant negative manner. This demonstrates that integrins and their ligands modulate functions in chondrocytes by influencing the synthesis of a pleiotropic cytokine such as IL-1β, IL-6 and IL-8, and signaling molecules such as NO and PGE₂, which have diverse catabolic functions. This study demonstrated the role of the extracellular matrix as a media for communication between the chondrocyte and the cartilage matrix. This is supported by compelling evidence in the literature that suggests that mechanical strain and overloading of cartilage or chondrocytes may influence integrin function (Daniels et al, 1991). This is further supported by Storgard et al, 1999, who reported that peptides that mimic the action of anti-3 monoclonal antibody, LM609, inhibit angiogenesis of the synovium in antigen-induced inflammatory arthritis in rabbits.

There are a variety of diseases in which inflammatory mediators, such as nitric oxide, IL-1β, IL-6, IL-8, PGE₂ and/or MMPs, are involved, including rheumatoid arthritis. Thus, methods which regulate these inflammatory mediators in a patient ameliorate symptoms of the disease and, depending upon the disease, can contribute to cure of the disease.

These diseases and conditions include, but are not limited to,

multiple sclerosis

type I diabetes

giant cell arthritis

systemic lupus erythematosus

Sjögren's Syndrome

rheumatoid arthritis

osteoarthritis

atherosclerosis

ischemia

arthrosclerosis

HIV infection and AIDS

bacterial infection

respiratory distress syndrome (AM)

smokers (AM)

coal mine pneumonoconiosis (AM)

alcoholic cirrhosis

cuprophane hemodialysis

cardiopulmonary bypass

chronic hepatitis B

thermal injury (burns)

reticulohistocytosis

sarcoidosis

tuberculosis

obstructive jaundice

Paget's Disease

osteomalacia

Kawasaki's Disease

inflammatory bowel disease

schistosoma infection

periodontal disease (periodontitis)

pancreatitis

renal dysfunction

Alzheimer's Disease

atopic dermatitis

respiratory viral infection

scleroderma

cerebral malaria

uveitis

skin diseases

chronic prostatitis

osteoporosis

hepatic fibrosis

host versus graft disease

proliferation of chronic myelogenous leukemia cells

organ transplantation

local inflammatory lesions

acute phase response

septic shock

vasculitis

The patient treated in the present invention in its many embodiments is preferably a human patient, although it is to be understood that the principles of the invention indicate that the invention is effective with respect to all mammals, which are intended to be included in the term “patient.” In this context, a mammal is understood to include any mammalian species in which treatment of diseases associated with inflammatory mediators is desirable, particularly agricultural and domestic mammalian species.

In a related embodiment, the invention contemplates the practice of the method in conjunction with other therapies, such as conventional anti-inflammatory medications, including steroids, non-steroidal anti-inflammatory drugs (NSAIDS), and COX-1 and COX-2 inhibitors and immunosuppressants, such as cyclosporin. Additionally, the agents of the present invention can be administered along with medications, such as TNFα and methotrexate.

The present method for regulating inflammatory mediators in a host comprises administering to the host which inflammatory mediators are affecting adversely, or a host which is at risk for adverse effects from inflammatory mediators, with a composition comprising a therapeutically effective amount of an agent which is capable of affecting the binding of the integrin responsible for the inflammatory mediator to its natural ligand. This agent can be LM609 or a compound which mimics the activity of LM609. Thus, the method comprises administering to a patient a therapeutically effective amount of a physiologically tolerable composition containing an integrin antibody or other agent according to the present invention.

The dosage ranges for the administration of the integrin agent with respect to inflammatory mediators depend on the form of the agent and its potency, and are amounts large enough to produce the desired effect in which inflammation and the disease symptoms mediated by inflammation are ameliorated. The dosage should not be so large as to cause adverse side effects, such as hyperviscosity syndrome, pulmonary edema, congestive heart failure, and the like. Generally, the dosage will vary with the age, condition and sex of the patent, as well as with the extent of the disease in the patient. Dosages can readily be determined by one of skill in the art. The dosage can also be adjusted by the individual physician in the event of any complication.

Regulation of inflammatory mediators can be measured in situ by immunochemistry or by any other methods known to one skilled in the art. Thus, one skilled in the art can readily determine if a compound has activity that mimics that of LM609. For purposes of the present invention, a compound that “substantially” mimics the activity of LM609 is one that has at least 50% of the activity, and preferably at least 75% of the activity, of LM609.

While the agents affecting integrin binding have been described as monoclonal antibodies or molecules having the antigen-binding portion of the antibody, these agents can take the form of an integrin mimetic or fragment thereof. Because the agent can be any one of these different types of molecules, it should be appreciated that the potency and, therefore, the expression of a “therapeutically effective” amount, can vary. However, one skilled in the art can readily assess the potency of a candidate integrin-affecting agent according to the present invention. These are described in Brooks et al, U.S. Pat. No. 5,753,230, the entire contents of which are hereby incorporated by reference.

Potency of an integrin-affecting agent can be measured by a variety of means, including inhibition of binding of a natural ligand to the integrin and similar assays.

A preferred integrin-affecting agent has the ability to substantially inhibit binding of a natural ligand, such as fibrinogen, vibronectin, osteopontin, von Willebrand factor or vitronectin, to the integrin in solution at agent concentrations of less than 0.5 μM, preferably less than 0.1 AM, and preferably less than 0.05 AM. By “substantially” is meant that at least a 50% reduction in binding of fibrinogen is observed by inhibition in the presence of the integrin-affecting agent, and a 50% inhibition is referred to herein as an IC₅₀ value.

A more preferred integrin-affecting agent exhibits selectivity to the integrin of interest over other integrins. Thus, a preferred αᵥβ₃-affecting agent substantially inhibits fibrinogen binding to αᵥβ₃ but does not substantially inhibit binding of fibrinogen to another integrin, such as αᵥβ₁, αᵥβ₅ or α_{IIB}β₃. Particularly preferred is an αᵥβ₃-affecting agent that exhibits a 10-fold to 100-fold lower IC₅₀ activity at inhibiting fibrinogen binding to αᵥβ₃ compared to the IC₅₀ activity at inhibiting fibrinogen binding to another integrin.

A therapeutically effective amount of integrin-affecting agent of the present invention in the form of a monoclonal antibody or fragment thereof, or other molecule having an antigen-binding portion of the antibody, is typically an amount such that when administered in a physiologically tolerable composition is sufficient to achieve a plasma concentration of from about 0.01 μg/ml to about 100 μg/ml, preferably from about 1 μg/ml to about 5 μg/ml. Stated differently, the dosage can vary from about 0.1 mg/kg to about 300 mg/kg, preferably from about 0.2 mg/kg to about 200 mg/kg, and most preferably from about 0.5 mg/kg to about 20 mg/kg in one or more dose administrations daily, for one or several days.

A therapeutically effective amount of an integrin-affecting agent in the form of a polypeptide is typically an amount of a polypeptide such that when administered in a physiologically tolerable composition is sufficient to achieve a plasma concentration of from about 1 μg/ml to about 200 μg/ml, preferably from about 1 μg/ml to about 150 μg/ml. Based upon a polypeptide having a mass of about 500 grams per mole, the preferred plasma concentration in molarity is from about 2 AM to about 5 mM, and preferably from about 100 AM to about 1 mM polypeptide antagonist. Stated differently, the dosage per body weight can vary from about 0.1 mg/kg to about 300 mg/kg, and preferably from about 0.2 mg/kg to about 200 mg/kg in one or more dose administrations daily, for one or over the course of several days.

The integrin-affecting agents of the present invention can be administered parenterally by injection or by gradual infusion over time. Although the tissue to be treated can typically be accessed in the body by systemic administration and, therefore, most often treated by intravenous administration of therapeutic compositions, other tissues and delivery are contemplated where there is a likelihood that the tissue targeted contains the target molecule. Thus, the integrin-affecting agents of the present invention can be administered intravenously, intraperitoneally, intramuscularly, subcutaneously, intracavity, transdermally, and can be delivered by peristaltic means.

The therapeutic compositions containing an integrin-affecting agent according to the present invention are conventionally-administered intravenously, as by injection of a unit dose. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier or vehicle.

Alternatively, LM609 can be administered by gene therapy techniques in which the LM609 is produced in the body in the location at which it is needed, or systemically, by genetically engineered cells.

One example of using gene therapy to produce LM609 is for treating leukemia and the inflammation associated therewith. The gene for LM609 is inserted into the bone marrow using conventional gene therapy techniques, after which the LM609 is expressed to control the inflammation.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The present invention contemplates therapeutic compositions useful for practicing the therapeutic methods described herein. Therapeutic compositions of the present invention contain a physiologically tolerable carrier together with an integrin-affecting agent as described herein, dissolved or dispersed therein as an active ingredient. In a preferred embodiment, the therapeutic integrin-affecting agent is not immunogenic when administered to a patient for therapeutic purposes.

The active ingredient(s) can be mixed with excipients which are pharmaceutically acceptable and compatible with the active ingredient and in amounts suitable for use in the therapeutic methods described herein. Suitable excipients are, for example, water, saline, dextrose, glycerol, ethanol, or the like and combinations thereof. In addition, if desired, the composition can contain minor amounts of auxiliary substances such as wetting and emulsifying agents, pH buffering agents, and the like, which enhance the effectiveness of the active ingredient.

The therapeutic compositions of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamine ethanol, histidine, procaine, and the like.

Integrin-affecting agents for regulating inflammatory mediators can be in the form of polypeptides, synthetic compounds or other types of proteins. A polypeptide integrin-affecting agent can have the sequence characteristics of either the natural ligand of the integrin of interest or of the integrin of interest itself at the region involved in integrin-ligand interaction and exhibits integrin-affecting agent activity as described herein. A preferred integrin-affecting agent peptide corresponds in sequence to the natural ligand.

Preferred polypeptides have a sequence corresponding to the amino acid residue sequence of the active region of a natural ligand of an integrin, such as fibrinogen, vitronectin, von Willebrand factor, laminin, thrombospondin, and the like ligands. The sequences of these ligands are well known. Thus, an integrin-affecting agent can be derived from any of the natural ligands, although fibrinogen and vitronectin are preferred.

A particularly preferred integrin-affecting peptide preferably inhibits integrin binding to its natural ligand(s) when compared to other integrins. These specific peptides are particularly preferred, at least because the specificity for the integrin of interest reduces the incidence of undesirable side effect, such as inhibition of other integrins. The identification of preferred integrinaffecting peptides having selectivity for the integrin of interest can readily be identified in a typical inhibition of binding assay, such as standard ELISA assay.

It should be understood that a subject polypeptide need not be identical to the amino acid residue sequence of the integrin of interest, as long as it includes the required sequence and is able to function as an integrin-affecting agent in an assay.

A subject agent includes any analog, fragment, or chemical derivative of a polypeptide which is an integrin-affecting agent. Therefore, the polypeptide can be subject to various changes, substitutions, insertions, and deletions where such changes provide for advantages in its use. In this regard, an integrin polypeptide of this invention corresponds to, rather than is identical to, the sequence of a recited peptide where one or more changes are made and it retains the ability to function as an integrin-affecting agent in one or more of the assays that can be used to test for integrin-affecting activity.

Thus, an agent polypeptide for use in the present invention can be in any variety of forms of peptide derivatives, including amides, conjugates the proteins, cyclized peptides, polymerized peptides, analogs, fragments, chemically modified peptides, and the like derivatives.

The term “analog” includes any polypeptide having an amino acid residue sequence substantially identical to a known agent which affects for an integrin in which one or more residues have been conservatively substituted with a functionally similar residue and which displays the integrin-affecting activity as described herein. Examples of conservative substitution include substituting one non-polar (hydrophobic) residue, such as isoleucine, valine, leucine or methionine for another; substituting one polar (hydrophilic) residue for another, such as between arginine and lysine, between glutamine and asparagine, or between glycine and serine; substituting one basic residue, such as lysine, arginine or histidine for another; or substituting one acidic residue, such as aspartic acid or glutamic acid for another.

The phrase “conservative substitution” also includes the use of a chemically derivatized residue in place of a non-derivatized residue provided that the polypeptide displays the requisite inhibiting activity.

“Chemical derivative” refers to a subject polypeptide having one or more re-sidues chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups, or formyl groups. Free carboxy groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-imbenzylhistidine. Also included as chemical derivatives are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides of the present invention also include any polypeptide having one or more additions and/or deletions or residues relative to the sequence of a polypeptide whose sequence is shown herein, as long as the requisite activity is maintained.

The term “fragment” refers to any polypeptide having an amino acid residue shorter than the amino acid residue of the naturally-occurring peptide but which retains its integrin-affecting activity.

When a polypeptide of the present invention has a sequence that is not identical to the sequence of an integrin natural ligand, it is typically because one or more conservative of non-conservative substitutions have been made. Usually no more than about 30 number percent, and preferably no more than 10 number percent of the amino acid residues are substituted. Additional residues may also be added at either terminus of a polypeptide for the purpose of providing a linker by which the polypeptide can be conveniently affixed to a label or solid matrix, or carrier.

Any peptide of the present invention can be used in the form of a pharmaceutically acceptable salt thereof.

A “variant” of the polypeptide according to the present invention refers to a molecule which is substantially similar to either the entire peptide or a fragment thereof. Variant peptides may be conveniently prepared by direct chemical synthesis of the variant peptide, using methods well known in the art.

Alternatively, amino acid sequence variants of the polypeptide can be prepared by mutations in the DNAs which encode the synthesized polypeptide derivatives. Such variants include, for example, deletions from, or insertions or substitutions of, or residues within the amino acid sequence. Any combination of deletion, insertion, and substitution may also be made to arrive at the final construct, provided that the final construct possesses the desired activity. Obviously, the mutations that will be made in the DNA encoding the variant peptide must not alter the reading frame and preferably will not create complementary regions that could produce secondary mRNA structure (cf. European Patent Publication No. EP 75,444).

At the genetic level, these variants ordinarily are prepared by site-directed mutagenesis of nucleotides in the DNA encoding the peptide molecule, thereby producing DNA encoding the variant, and thereafter expressing the DNA in recombinant cell culture. The variants typically exhibit the same qualitative biological activity as the non-variant peptide.

The types of substitutions which may be made in the polypeptide according to the present invention may be based on analysis of the frequencies of amino acid changes between a homologous protein of different species. Based upon such analysis, conservative substitutions may be defined herein as exchanges within one of the following five groups:

I. Small, aliphatic, non-polar or slightly polar residues:

Ala, Ser, Thr, Pro, Gly

II. Polar, negatively-charged residues and their amides:

Asp, Asn, Glu, Gln

III. Polar, positively-charged residues:

His, Arg, Lys

IV. Large, aliphatic non-polar residues:

Met, Leu, Ile, Val, Cys

V. Large aromatic residues:

Phe, Try, Trp

Within the foregoing groups, the following substitutions are considered to be “highly conservative”:

Asp/Glu

His/Arg/Lys

Phe/Tyr/Trp

Met/Leu/Ile/Val

Semi-conservative substitutions are defined to be exchanges between two of groups (I)-(IV) above which are limited to supergroup (A), comprising (I), (II), and (III) above, or to supergroup (B), comprising (IV) and (V) above. Substitutions are not limited to the genetically encoded or even the naturally-occurring amino acids. When the epitope is prepared by peptide synthesis, the desired amino acid may be used directly. Alternatively, a genetically encoded amino acid may be modified by reacting it with an organic derivatizing agent that is capable of reacting with selected side chains or terminal residues.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-β-(5-imidazoyl)propionic acid, chloroacetyl phosphate, N-alkylmaleimides, 3-nitro-2-pyridyl disulfide, methyl-2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Parabromophenacyl bromide is also useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino acid-containing residues include imidoesters such as methyl picolinimidate; pyridoxal phosphate; pyridoxal; chloroborohydride; trinitrobenzenesulfonic acid; O-methyliosurea; 2,4-pentanedione; and transaminase-catalyzed reaction with glyoxylate.

Arginyl residues are modified by reaction with one or several conventional reagents, among them phenylglyoxal; 2,3-butanedione; and ninhydrin. Derivatization of arginine residues requires that the reaction be performed in alkaline conditions because of the high pKa of the guanidine functional group. Furthermore, these reagents may react with the groups of lysine, as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and e-nitro derivatives, respectively.

Carboxyl side groups (aspartyl or glutamyl) are selectively modified by reaction with carbodiimides (R′N—C—N—R′) such as 1-cyclohexyl-3-[2-morpholinyl-(4-ethyl)]carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

The polypeptides of the present invention can be synthesized by any of the techniques known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like.

In one embodiment the integrin-affecting agents are monoclonal antibodies which immunoreact with the integrin of interest and inhibit binding of the integrin to its natural ligand. The invention also includes cell lines which produce the antibodies, methods for producing the cell lines, and methods for producing the monoclonal antibodies.

It should be understood that, when the term “antibody” or “antibodies” is used herein, this is intended to include intact antibodies, such as monoclonal antibodies (mAbs), as well as proteolytic fragments thereof, such as the Fab or F(ab′)₂ fragments. Furthermore, the DNA encoding the variable region of the antibody can be inserted into other antibodies to produce chimeric antibodies (see, for example, U.S. Pat. No. 4,816,567). Single-chain antibodies can also be produced and used. Single-chain antibodies can be single-chain composite polypeptides having antigen-binding capabilities and comprising a pair of amino acid sequences homologous or analogous to the variable regions of an immunoglobulin light and heavy chain (linked V_{H}-V_{L} or single-chain F_{V}). Both V_{H} and V_{L} may copy natural monoclonal antibody sequences or one or both of the chains may comprise a CDR-FR construct of the type described in U.S. Pat. No. 5,091,513. The separate polypeptides analogous to the variable regions of the light and heavy chains are held together by a polypeptide linker. Methods of production of such single-chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815.

Polyclonal antibodies are heterogeneous populations of antibody molecules derived from the sera of animals immunized with an antigen. A monoclonal antibody contains a substantially homogeneous population of antibodies specific to antigens, which population contains substantially similar epitope binding sites. MAbs may be obtained by methods known to those skilled in the art, including Kohler et al (1975); U.S. Pat. No. 4,376,110; Ausubel et al (1987-1994); Harlow et al (1988); and Coligan et al (1993), the contents of which references are incorporated entirely herein by reference. Such antibodies may be of any immunoglobulin class, including IgG, IgM, IgE, IgA, GILD, and any subclass thereof. A hybridoma producing a mAb of the present invention may be cultivated in vitro, in situ, or in vivo.

Accordingly, mAbs generated against LM609 and related proteins of the present invention may be used to induce anti-Id antibodies in suitable animals, such as BALB/c mice. Spleen cells from such immunized mice are used to produce anti-Id hybridomas secreting anti-Id mAbs. Further, the anti-Id mAbs can be coupled to a carrier, such as keyhole limpet hemocyanin (KLH), and used to immunize additional BALB/c mice. Sera from these mice will contain anti-anti-Id antibodies that have the binding properties of the original mAb specific for a LM609 epitope. The anti-Id mAbs thus have their own idiotypic epitopes, or “idiotopes”, which are structurally similar to the epitope being evaluated.

The term “antibody” is also meant to include both intact molecules, as well as active fractions thereof, such as, for example, Fab and F(ab′)₂₁ which are capable of binding antigens. Fab and F(ab′)₂ fragments lack the Fc fragment of intact antibody, clear more rapidly from the circulation, and may have less non-specific tissue binding than an intact antibody (Wahl et al, 1983).

It will be appreciated that the Fab and F(ab′)₂ and other fragments of the antibodies useful in the present invention may be used for the detection and quantification of LM609 and related proteins according to the methods disclosed herein for intact antibody molecules. Such fragments are typically produced by proteolytic cleavage, using enzymes such as papain (to produce Fab fragments) or pepsin (to produce F(ab′)₂ fragments).

An antibody is said to be “capable of binding” a molecule if it is capable of specifically reacting with the molecule to thereby bind the molecule to the antibody. The term “epitope” is meant to refer to that portion of any molecule capable of being bound by an antibody which can also be recognized by that antibody. Epitopes or “antigenic determinants” usually consist of chemically active surface groupings of molecules, such as amino acids or sugar side chains and have specific three-dimensional structural characteristics, as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

A “molecule which includes the antigen-binding portion of an antibody” is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, and chimeric humanized or single-chain antibodies incorporating such reactive fraction, as well as any other type of molecule in which such antibody reactive fraction has been physically inserted or molecules developed to deliver therapeutic moieties by means of a portion of the molecule containing such a reactive fraction. Such molecules may be provided by any known technique, including, but not limited to, enzymatic cleavage, peptide synthesis or recombinant techniques, such as phage display libraries.

Monoclonal antibodies (mAbs) are a substantially homogeneous population of antibodies to specific antigens. MAbs may be obtained by methods known to those skilled in the art. See, for example Kohler et al (1975); U.S. Pat. No. 4,376,110; Ausubel et al (1987-94); Harlow et al (1988); and Coligan et al (1993). Such antibodies may be of any immunoglobulin class including IgG, IgM, IgE, IgA, IgD and any subclass thereof. The hybridoma producing the mAbs may be cultivated in vitro or in vivo. High titers of mabs can be obtained in in vivo production where cells from the individual hybridomas are injected intraperitoneally into pristane-primed BALB/c mice to produce ascites fluid containing high concentrations of the desired mAbs. MAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Chimeric antibodies are primarily used to reduce immunogenicity in application and to increase yields in production, for example, where murine mabs have higher yields from hybridomas but higher immunogenicity in humans, such that human/murine chimeric (humanized) mabs are used. Chimeric antibodies and methods for their production are known in the art (Cabilly et al, 1984; Morrison et al, 1984; European Patent Application 125023; Neuberger et al, 1985; European Patent Application 171496; European Patent Application 173494; PCT Application WO 8601533; European Patent Application 184187; European Patent Application 173494; Sahagan et al, 1986; WO 9702671; Liu et al, 1987; Sun et al, 1987; Better et al, 1988; and Harlow et al, 1998).

An anti-idiotypic (anti-Id) antibody is an antibody which recognizes unique determinants generally associated with the antigen-binding site of an antibody. An anti-Id antibody can be prepared by immunizing an animal of the same species and genetic type (e.g., mouse strain) as the source of the mAb with the mAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880.

The anti-Id antibody may also be used as an “immunogen” to induce an immune response in yet another animal, producing a so-called anti-anti-Id antibody. The anti-anti-Id may bear structural similarity to the original mAb which induced the anti-Id. Thus, by using antibodies to the idiotypic determinants of a mAb, it is possible to identify clones expressing antibodies of identical specificity.

Many laboratories have used in vitro antibody phage display libraries to screen or “pan” for phage displayed antibodies against specific antigens, and a representative though certainly not exhaustive, list of citations include Sawyer et al (1977); Waters et al (1997); Figini et al (1998); Chowdhury et al (1997); Pfistermueller et al (1996); Kakinuma et al (1997); Iba et al (1997); Pereira et al (1997a and 1997b); Siegel et al (1997); and Osbourn et al (1996). Some laboratories have established whole cell based systems for a screening procedure for the detection and isolation of cell surface antigens and procedures for optimizing the capture of cell surface specific antibodies using antibody phage display (Watters et al, 1997; Chowdhury et al, 1997; Pereira et al, 1997a and 1997b; Siegel et al, 1997). For instance, Siegel et al (1997) optimized the capture of cell surface specific human antibodies using phage display and minimized the binding of irrelevant phage displayed antibodies using a simultaneous positive and negative selection strategy.

A specific example of a method for panning antibodies against cell surface antigens using in vitro antibody phage display is a method derived from Palmer et al (1997), where a single pot of human Fv semi-synthetic filamentous phage display library is to be constructed in the pHEN1 vector according to the procedure of Nissim et al (1994). The library will be rescued with VC3M13 helper phage (Stratagene, La Jolla, Calif.), and the phage will be purified using polyethylene glycol. For each round of selection for phage which bind to brain microcapillary endothelial cells (BMEC), approximately 1013 transducing units of phage in PBS with 5% milk powder (for non-specific blocking) will be added to target BMEC and incubated overnight at 4@C. Cells will then be washed with PBS, 1% albumin, to remove unbound phage. Bound phage will be eluted from BMEC by adding 300 μl of 76 mM citric acid in PGS (pH 2.5), and the fluid neutralized with 400 μl 1M Tris-HCl, pH 7.4. The phage will be subsequently expanded overnight in E. coli TG1 cells.

Phage particles will be enriched for specific high-affinity antigen binding phage through a further five rounds of binding to BMEC, and screening for binding to a panel of cell types, such as dermal microcapillaries, foreskin microcapillaries, umbilical vein endothelial cells, aorta and standard human cell lines derived from cornea, keratinocytes, kidney, etc., to determine cell and tissue specificity. Only those phage which exclusively bind BMEC will be used for further experiments. The plasmid carried by the selected phage which encode the Fv segment(s) will be isolated, characterized and cloned for expression in bacterial host cells to produce a soluble Fv segment(s) that can be purified and used for derivatization with cross-linkers for drug delivery.

A monoclonal antibody of the present invention comprises antibody molecules that (1) immunoreact with isolated integrin of interest and (2) inhibit fibrinogen binding to the integrin of interest. Preferred monoclonal antibodies include LM609.

The term “antibody or antibody molecule” in the various grammatical forms used herein refers to a population of immunoglobulin molecules and/or immunologically active portions of molecules that contain an antibody combining site or paratope. An “antibody combining site” is that structural portion of an antibody molecule comprised of heavy and light chain variable and hypervariable regions that specifically binds antigen.

Exemplary antibodies for use in the present invention are intact immunoglobulin molecules, substantially intact immunoglobulin molecules, and those portions of an immunoglobulin molecule that contain the paratope, including those portions known in the art as Fab, Fab′, F(ab′)₂, and F(v), and also referred to as antibody fragments.

In another preferred embodiment, the invention contemplates a truncated immunoglobulin molecule comprising a Fab fragment derived from a monoclonal antibody of the invention. The Fab fragment, lacking Fc receptor, is soluble and affords therapeutic advantages in serum half life, and diagnostic advantages in modes of using the soluble Fab fragment. The preparation of a soluble Fab fragment is generally known in the immunological arts and can be accomplished by a variety of methods.

The phrase “monoclonal antibody” refers to a population of antibody molecules that contain only one species of antibody combining site capable of immunoreacting with a particular epitope. A monoclonal antibody, thus, typically displays a single binding affinity for any epitope with which it immunoreacts. A monoclonal antibody may, therefore, contain an antibody molecule having a plurality of antibody combining sites, each immunospecific for a different epitope, e.g., a bispecific monoclonal antibody.

It is possible to determine, without undue experimentation, if a monoclonal antibody has the same, i.e., equivalent, specificity, i.e., immunoreactive characteristics, such as a monoclonal antibody of this invention by ascertaining whether the former prevents the latter from binding to a pre-selected target molecule. If the monoclonal antibody being tested competes with the monoclonal antibody of the invention, as shown by a decrease in binding by the monoclonal antibody of the invention in standard competition assays for binding to the target molecule when present in the solid phase, then it is likely that the two monoclonal antibodies bind to the same, or a closely related, epitope.

Use of the term “having the binding specificity of” indicates that equivalent monoclonal antibodies exhibit the same or similar immunoreaction (binding) characteristics and compete for binding to a preselected target molecule.

To determine whether a molecule is an antagonist for an integrin of interest, direct binding to a natural ligand of the integrin of interest can be assayed. The assay can also be used to identity molecules which exhibit specificity for the integrin of interest and do not inhibit natural ligands from binding other integrins.

The present invention also provides a method for assaying for integrin-affecting properties, which can be used for screening anti-inflammatory drugs.

To test the anti-inflammatory effects of a compound, bovine chondrocytes, endothelial cells, or rabbit synovial cells were incubated in RPM11640 or Hams F12 medium or any other basal medium in the absence of any supplements or serum. Alternatively arthritis-affected cartilage was incubated in RPM11640 or Hams F12 medium or any other basal medium in the absence of any supplements or serum with 0.1% human albumin. The cells were stimulated with various modulators, including IL-1, LPS, etc. and blocked with LM609. The amounts of NO, PGE₂, IL-3, IL-6, and IL-8 were assayed at 72 hours post stimulation using a variety of methods. The compounds are identified in their anti-inflammatory effects by the amount of NO, PGE2, IL-1, IL-6, or IL-8 detected.

As used herein, the term “muteins” refers to analogs of LM609 proteins in which one or more of the amino acid residues of the natural LM609 proteins or their active fractions are replaced by different amino acid residues, or are deleted, or one or more amino acid residues are added to the natural sequence of the LM609 protein(s), without changing substantially the activity of the resulting products as compared with wild type LM609 protein(s). These muteins are prepared by known synthesis and/or by site-directed mutagenesis techniques, or any other known technique suitable therefor.

Any such mutein preferably has a sequence of amino acid sufficiently duplicative of those of LM609 protein(s), such as to have substantially similar activity to the LM609 protein(s). This activity can be measured by noting the activity of the mutein on tumor cells. “Substantially similar” means that the mutein affects the growth of tumor cells in the same manner as LM609 protein(s), even if the degree of the effect is smaller or greater than that of naturally occurring LM609.

In a preferred embodiment, any such mutein has at least 40% identity or homology with the sequence of any of the LM609 protein(s). More preferably, it has at least 50%, at least 60%, at least 70%, at least 80%, or most preferably, at least 90% identity or homology thereto.

Muteins of LM609 protein(s) which can be used in accordance with the present invention, or nucleic acids coding therefor, include a finite set of substantially corresponding sequences as substitution peptides or polynucleotides which can be routinely obtained by one of ordinary skill in the art, without undue experimentation, based on the teachings and guidance presented herein. For a detailed description of protein chemistry and structure, see Schulz et al (1978) and Creighton (1983), which are hereby incorporated by reference. For a presentation of nucleotide sequence substitutions, such as codon preferences, see Ausubel et al (1987-1994) at Sections A.1.1-A.1.24, and Sambrook et al (1989) at Appendices C and D.

Preferred changes for muteins in accordance with the present invention are what are known as “conservative” substitutions. Conservative substitutions of LM609 polypeptides or proteins may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. For a discussion of this, see Anfinsen (1973). Proteins and muteins produced by such deletions and/or insertions come within the purview of the present invention.

Examples of productions of amino acid substitutions in proteins which can be used for obtaining muteins of LM609 proteins or polypeptides for use in the present invention include any known method steps, such as described in U.S. Pat. Nos. Re 33,632, 4,959,314, 4,588,585 and 4,737,462 to Mark et al; U.S. Pat. No. 5,116,943, to Koths et al; U.S. Pat. No. 4,965,195 to Namen et al; U.S. Pat. No. 4,879,111 to Chong et al; and U.S. Pat. No. 5,017,691 to Lee; and lysine substituted proteins described in U.S. Pat. No. 4,904,584, to Shaw et al.

In another preferred embodiment of the present invention, any mutein of LM609 proteins or polypeptides has an amino acid sequence essentially corresponding to that of LM609 protein or polypeptide. The term “essentially corresponding to” is intended to comprehend proteins with minor changes to the sequence of the natural protein which do not affect the basic characteristics of the natural proteins, particularly insofar as their ability to modulate the inflammatory response is concerned. The types of changes which are generally considered to fall within the “essentially corresponding to” language are those which would result from conventional mutagenesis techniques of the DNA encoding these proteins, resulting in a few minor modifications, and screening for the desired activity in the manner discussed above. Muteins in accordance with the present invention include proteins encoded by a nucleic acid, such as DNA or RNA, which hybridizes to DNA or RNA which encodes LM609 proteins in accordance with the present invention under stringent conditions. The invention also includes such nucleic acid which is also useful as a probe in identifying and purifying the desired nucleic acid. Furthermore, such nucleic acid would also be a prime candidate to determine whether it encodes a polypeptide which retains the functional activity of the LM609 of the present invention. The term “stringent conditions” refers to hybridization and subsequent washing conditions which those of ordinary skill in the art conventionally refer to as “stringent.” See Ausubel et al (1987-1994) and Sambrook et al (1989). Without limitations, examples of stringent conditions include washing conditions 12-20° C. below the calculated Tm of the hybrid under study in, e.g., 1×SSC and 0.5% SDS for 5 minutes, 2×SSC and 0.1 SDS for 15 minutes; 0.1×SSC and 0.5% SDS at 37° C. for 30-60 minutes and then a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC, cf. Ausubel (1987-1994)

The term “fused protein” refers to a polypeptide comprising LM609 or active fractions thereof fused with another protein which, e.g., has extended residence time in body fluids. Thus, LM609 may be fused to another protein, polypeptide, or the like, e.g., an immunoglobulin or a fragment thereof. Additionally, the other protein or polypeptide may be one which preferentially delivers the LM609 to the active site.

The term “salts” herein refers to both salts of carboxyl groups and to acid addition salts of amino groups of LM609, their active fractions, muteins, or fused proteins thereof. Salts of a carboxyl group may be formed by means known in the art and include inorganic salts, such as sodium, calcium, ammonium, ferric, or zinc salts and the like, and salts with organic bases, such as those formed, for example, with amines, including triethanolamine, arginine, lysine, piperidine, procaine, and the like. Acid addition salts include, for example, salts with mineral acids such as, for example, hydrochloric acid or sulfuric acid, and salts with organic acids such as, for example, acetic acid or oxalic acid. Of course, any such salts must have substantially similar activity to LM609 or their active fractions.

“Functional derivatives” as used herein covers derivatives of LM609 proteins or their active fractions and muteins and fused proteins which may be prepared from the functional groups which occur as-side chains on the residues of the N- or C-terminal groups, by means known in the art, and are included in the invention as long as they remain pharmaceutically acceptable, i.e., they do not destroy the activity of the protein which has activity substantially similar to that of LM609, and do not confer toxic properties to the protein. These derivatives may, for example, include polyethylene glycol side chains, which may mask antigenic sites and extend the residence of LM609 in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g., alkanoyl or carbocyclic aroyl groups) or O-acyl derivatives of free hydroxyl groups (such as that of seryl or threonyl residues) formed with acyl moieties.

As “active fractions” of LM609, muteins, and fused proteins thereof, the present invention covers any fragment or precursors of the polypeptide chain of the protein molecules, or fused proteins containing any such fragment of LM609>, alone or together with associated molecules or residues linked thereto, e.g., sugar or phosphate residues, or aggregates of any of the above protein molecules, provided said fraction has substantially similar activity to LM609.

The present invention also includes antibodies against LM609, their muteins, fused proteins, salts, functional derivatives, and active fractions, as well as other compounds which mimic the activity of LM609.

In order to produce LM609 by genetic engineering techniques, LM609 is isolated and is then subjected to protein microsequencing. Internal sequences are reverse-translated into sense and antisense primers to which suitable restriction sites are added. Total RNA is purified from human cells and first strand cDNA is generated with reverse transcriptase, using either the antisense oligonucleotide mixture or oligo d(T) as a primer. The resulting cDNA fragment is then amplified in a polymerase chain reaction (PCR), using the combined sense and antisense degenerate primers. The PCR products are analyzed on a 3% agarose gel, and the specific oligonucleotide band is restriction-digested, cloned into pBluescript (Stratagene) and competent E. coli transfected with this vector. Several independent clones are then sequenced. The sequence of the region flanked by the sense and antisense degenerate primers is invariant and encodes the expected sequence from the above-mentioned LM609. An oligonucleotide sequence corresponding to this non-degenerate internal sequence is synthesized, end-labelled, and used for screening cDNA libraries.

DNA coding for the precursor of a truncated soluble form of LM609 is generated by PCR. The resulting PCR product is inserted into a mammalian expression vector and used for transfection of various mammalian cells, such as monkey COS cells. Such cells express high levels of biologically active recombinant LM609. Similarly, DNA coding for the entire precursor of LM609 is generated by PCR. The resulting PCR product is inserted into a mammalian expression vector and used for transfection of various mammalian cells, such as mouse NIH-3T3 cells. Such cells express high levels of human LM609.

In an alternative way of cloning the LM609 gene, a library of expression vectors is prepared by cloning DNA or, more preferably, cDNA (from a cell capable of expressing LM609 protein) into an expression vector. The library is then screened for members capable of expressing a protein which binds to anti-LM609 antibody, and which has a nucleotide sequence that is capable of encoding polypeptides that have the same amino acid sequence as LM609 protein, or fragments or muteins thereof. In this embodiment, DNA, or more preferably cDNA, is extracted and purified from a cell which is capable of expressing LM609 antigens. The purified cDNA is fragmentized (by shearing, endonuclease digestion, etc.) to produce a pool of DNA or cDNA fragments. DNA and cDNA fragments from this pool are then cloned into an expression vector in order to produce a genomic library of expression vectors whose members each contain a unique cloned DNA or cDNA fragment.

An “expression vector” is a vector which, due to the presence of appropriate transcriptional and/or translational control sequences, is capable of expressing a DNA or cDNA molecule which has been cloned into the vector and of thereby producing a polypeptide or protein. Expression of the cloned sequences occurs when the expression vector is introduced into an appropriate host cell. If a prokaryotic expression vector is employed, then the appropriate host cell would be any prokaryotic cell capable of expressing the cloned sequences. Similarly, if a eukaryotic expression vector is employed, then the appropriate host cell would be any eukaryotic cell capable of expressing the cloned sequences. Importantly, since eukaryotic DNA may contain intervening sequences, and since such sequences cannot be correctly processed in prokaryotic cells, it is preferable to employ cDNA from a cell which is capable of expressing adipogenic protein in order to produce a prokaryotic genomic expression vector library. Procedures for preparing cDNA and for producing a genomic library are disclosed by Sambrook et al (1989).

LM609 can also be produced by other types of recombinant cells such as prokaryotic cells, e.g., E. coli, or other eukaryotic cells, such as CHO, yeast, or insect cells. Methods for constructing appropriate vectors, carrying DNA that codes for LM609 and suitable for transforming (e.g., E. coli and yeast cells) or infecting insect cells in order to produce recombinant LM609 are well known in the art. For example, see Ausubel et al (1987-1994) and Sambrook et al (1989).

The present invention further relates to active muteins and fragments of LM609 and to fused proteins consisting of wild type LM609 or their active muteins or their active fractions fused to another polypeptide or protein and exhibiting a similar ability to affect the growth and survival of tumor cells.

DNA encoding LM609, their fragments, muteins, or fused protein, and the operably linked transcriptional and translational regulatory signals, are inserted into eukaryotic vectors which are capable of integrating the desired gene sequences into the host cell chromosome. In order to be able to select the cells which have stably integrated in introduced DNA into their chromosomes, one or more markers which allow for selection of host cells which contain the expression vector are used. The marker may provide for prototrophy to an auxotropic host, biocide resistance, e.g., antibiotics, or resistance to heavy metals, such as copper, or the like. The selected marker gene can either be directly linked to the DNA gene sequences to be expressed, or introduced into the same cell by cotransfection. Additional elements may also be needed for optimal synthesis of single-chain binding protein mRNA. These elements may include splice signals, as well as transcription promoters, enhancers, and termination signals.

For expressing LM609 proteins, their active fractions or derivatives, the DNA molecule to be introduced into the cells of choice is preferably incorporated into a plasmid or viral vector capable of autonomous replication in the recipient host.

Factors of importance in selecting a particular plasmid or viral vector include: the ease with which recipient cells that contain the-vector may be recognized and selected from those recipient cells which do not contain the vector; the number of copies of the vector which are desired in a particular host; and whether it is desirable to be able to “shuttle” the vector between host cells of different species. Preferred prokaryotic vectors include plasmids such as those capable of replication in E. coli, for example, pBR322, Co1E1, pSC101, pACYC 184, etc.; Bacillus plasmids, such as pC194, pC221, pT127, etc; Streptomyces plasmids, including pIJ101; Streptomyces; bacteriophages, such as C31; and Pseudomonas plasmids.

Preferred eukaryotic plasmids include BPV, vaccinia, SV40, 2-micron circles, etc. or their derivatives. Such plasmids are well known in the art.

Once the vector or DNA sequence containing the construct(s) has been prepared for expression, the expression vector may be introduced into an appropriate host cell by any of a variety of suitable means, such as transformation, transfection, lipofection, conjugation, protoplast fusion, electroporation, calcium phosphate precipitation, direct microinjection, etc.

Host cells to be used in the present invention may be either prokaryotic or eukaryotic. Preferred prokaryotic hosts include bacteria such as E. coli, Bacillus, Streptomyces, Pseudomonas, Salmonella, Serratia, etc. The most preferred prokaryotic host is E. coli. Under these conditions, the protein will not be glycosylated. The prokaryotic host must be compatible with the replicon and control sequences in the expression plasmid.

Preferred eukaryotic hosts, which produce glycosylated proteins, are mammalian cells, such as human, monkey, mouse, and Chinese hamster ovary cells, because they provide post-translational modification to protein molecules including correct folding, correct disulfide bond formation, as well as glycosylation at correct sites. Also, yeast cells and insect cells can carry out post-translational peptide modifications including high mannose glycosylation. A number of recombinant DNA strategies exist which use strong promoter sequences and high copy number of plasmids which can be used for producing the desired proteins in yeast and in insect cells. Yeast cells recognize leader sequences on cloned mammalian gene products and secrete peptides bearing leader sequences.

Strong promoters are the most preferred promoters of the present invention. Examples of such preferred promoters are those which recognize the T3, SP6 and T7 polymerase promoters; the PL promoter of bacteriophage lambda; the recA promoter and the promoter of the mouse metallothionein I gene. The most preferred promoter is one which is capable of recognizing the T7 polymerase promoter.

After the introduction of the vector, the host cells are grown in a selective medium, which selects for the growth of vector-containing cells. Expression of the cloned gene sequence(s) results in the product of LM609 proteins, fusion proteins, or muteins or fragments thereof. The expressed proteins are then isolated and purified by any conventional procedure involving extraction, precipitation, chromatography, electrophoresis, or the like, or by affinity chromatography, using anti-LM609 monoclonal antibodies immobilized on a gel matrix contained within a column. Crude preparations containing said recombinant LM609 are passed through the column whereby LM609 and their active fractions or derivatives are bound to the column by the specific antibody while the impurities pass through. After washing, the protein is eluted from the gel under conditions usually employed for this purpose, i.e., at a high or a low pH, e.g., pH 11 or 2.

Preferred methods for purifying and characterizing the proteins include sodium dodecyl sulfate polyacrylamide gel electrophoresis (SDS-PAGE) performed according to the method of Laemmli (1970) using 7.5% acrylamide gels with a constant ratio of 2.6% bisacrylamide/total acrylamide concentration. Protein samples are denatured at 100° C. for ten minutes in 20 mM Tris containing 3.3% glycerol, and bromophenol blue tracking dye with or without proteins with 0.05% R250 Coomassie brilliant blue in 25% isopropanol for two hours and destained for 24 hours in 20% methanol-7% acetic acid. As molecular weight markers, myosin (200 kDa), beta-galactosidase (116 kDa), phosphorylase B (97 kDa), BSA (66 kDa), and egg albumin (43 kDa) are used. Known modifications and variations of the described method are also contemplated within the scope of this invention.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Amin et al, “The expression and regulation of nitric oxide synthase in human osteoarthritis-affected chondrocytes: evidence for up-regulated neuronal nitric oxide synthase J. Exam Med. 182:2097-2102 (1995)

Amin et al, “Superinduction of cyclooxygenase-2 activity in human osteoarthritis-affected cartilage: influence of nitric oxide”, J. Clin. Inv. 99(6):1231-1237 (1997)

Anfinsen, C. B., “Principles that govern the folding of protein chains”, Science; 181(96):223-230 (1973).

Arner et al (1994)

Aplin et al, “Signal transduction and signal modulation by cell adhesion receptors: the role of integrins, cadherins, immunoglobulin-cell adhesion molecules, and selecting”, Pharmacological Reviews 50(2):197-263 (1998)

Attur (1999)

Ausubel et al, Eds., Current Protocols In Molecular Biology, Green Publishing Assoc., and Wiley Interscience (New York, 1987-1994)

Babic et al, “CYR61, a product of a growth factor-inducible immediate early gene, promotes angiogenesis and tumor growth”, Proc. Nat. Acad. Sci. USA 95(11):6355-6360 (1998)

Better et al, Science 240:1041-1043 (1988)

Beuerlein et al (1998)

Blystone et al, “Integrin alpha v beta 3 differentially regulates adhesive and phagocytic functions of the fibronectin receptor alpha 5 beta 1”, J. Cell. Biol.” 127(4):1129-1137 (1994)

Brooks et al, “Disruption of angiogenesis by PEX, a noncatalytic metalloproteinase fragment with integrin binding activity”, Cell 92(3):391-400 (1998)

Brooks et al, “Requirement of vascular integrin αᵥβ₃ for angiogenesis”, Science 264(5158):569-571 (1994)

Byzova et al, “Activation of αᵥβ₃ on vascular cells controls recognition of prothrombin”, J. Cell Biol. 143(7):2081-2092 (1998)

Cabilly et al, “Generation of antibody activity from immunoglobulin polypeptide chains produced in Escherichia coli.”, Proc. Nat. Acad. Sci. USA 81(11):3273-3277 (1984)

Cheresh et al, “Biosynthetic and functional properties of an Arg-Gly-Asp-directed receptor involved in human melanoma cell attachment to vitronectin, fibrinogen, and von Willebrand factor,” J. Biol. Chem. 262(36):17703-177011 (1987)

Cheresh, D. A., “Human endothelial cells synthesize and express an Arg-Gly-Asp-directed adhesion receptor involved in attachment to fibrinogen and von Willebrand factor”, Proc. Natl. Acad. Sci. 84(18):6471-6475 (1987)

Chowdhury et al, “Isolation of anti-mesothelin antibodies from a phage display library”, Mol. Immunol. 34(1):9-20 (1997)

Coligan et al, Eds., Current Protocols in Immunology, Green Publishing Assoc., and Wiley Interscience (New York, 1993)

Creighton, T. E., Proteins: Structure and Molecular Properties, W. H. Freeman & Co. (San Francisco, 1983)

Daniels et al (1991)

Dattur et al, Proc. Amer. Assoc. Physicians 110:1 (1998)

Denhardt and Guo, (ID:509), “Osteopontin: a protein with diverse functions”, FASEB J. 7(15):1475-1482 (1993)

Denhardt, Lopez et al, (ID:510), “Osteopontin-induced modifications of cellular functions”, Ann. NY Acad. Sci. 760:127-142 (1995).

Denhardt and Noda, (ID:1682), “Osteopontin expression and function: role in bone remodeling”, J. Cell Biochem. Suppl. 30-31:92-102 (1998).

Diaz-Gonzalez et al, “Trans-dominant inhibition of integrin function”, Mol. Biol. Cell. 7(12):1939-1951 (1996).

DiCesare et al, “Up-regulation of inducible nitric oxide synthase and production of nitric oxide by the Swarm rat and human chondrosarcoma”, J. Orthop. Res. 16(6):667-674 (1998)

Duong et al, “PYK2 in osteoclasts is an adhesion kinase, localized in the sealing zone, activated by ligation of αᵥβ₃ integrin, and phosphorylated by src kinase”, J. Clin. Invest. 102(5):881-892 (1998)

Eliceiri et al, “Integrin αᵥβ₃ requirement for sustained mitogen-activated protein kinase activity during angiogenesis”, J. Cell. Biol. 140(5):1255-1263 (1998)

Figini et al, “Panning phage antibody libraries on cells: isolation of human Fab fragments against ovarian carcinoma using guided selection”, Cancer Res. 58(5):991-996 (1998)

Georgescu et al, “Proline isomerization-independent accumulation of an early intermediate and heterogeneity of the folding pathways of a mixed alpha/beta protein, Escherichia coli thioredoxin”, Biochemistry 37(28):10286-10297 (1998)

Gilliam et al (1993)

Gladson et al (1995)

Grantham, “Amino acid difference formula to help explain protein evolution,” Science 185(154):862-864 (1974)

Gu et al, “Characterization of bone marrow laminins and identification of α₅-containing laminins as adhesive proteins for multipotent hematopoietic FDCP-Mix cells.”, Blood 93(8):2533-2542 (1999).

Harlow et al, Antibodies: A Laboratory Manual, Cold Spring Harbor Laboratory (1988)

Hermann et al, “The vitronectin receptor and its associated CD47 molecule mediates proinflammatory cytokine synthesis in human monocytes by interaction with soluble CD23”, J. Cell Biol. 144(4):767-775 (1999).

Hocking et al, “Activation of distinct αᵥβ₃-mediated signaling pathways by fibronectin's cell adhesion and matrix assembly domains”, J. Cell. Biol. 141(1):241-253 (1998).

Hodivala-Dilke et al, “Novel roles for α₃β₁ integrin as a regulator of cytoskeletal assembly and as a trans-dominant inhibitor of integrin receptor function in mouse keratinocytes”, J. Cell Biol. 142(5):1357-1369 (1998)

Homandberg et al, “Fibronectin fragments cause chondrolysis of bovine articular cartilage slices in culture”, J. Biol. Chem. 267(6):3597-3604 (1992)

Homandberg et al, “Fibronectin-fragment-induced cartilage chondrolysis is associated with release of catabolic cytokines”, Biochem. J. 321(pt 3):751-757 (1997)

Hraeus et al (1999)

Huynh-Do et al, “Surface densities of ephrin-B1 determine EphB1-coupled activation of cell attachment through αᵥβ₃ and αᵥβ₁ integrins”, EMBO J. 18(8):2165-2173 (1999)

Hwang, Lopez et al, (ID:489), “Osteopontin inhibits induction of nitric oxide synthase gene expression by inflammatory mediators in mouse kidney epithelial cells”, J. Biol Chem. 269(1):711-715 (1994).

Iba et al, “Comparison of strategies for the construction of libraries of artificial antibodies”, Immunol. Cell Biol. 75(2):217-221 (1997)

Kakinuma et al, “Insight into screening immunoglobulin gene combinatorial libraries in a phage display vector: a tale of two antibodies”, Autoimmunity 25(2):73-84 (1997)

Kanbey (1991)

Kohler et al, “Continuous cultures of fused cells secreting antibody of predefined specificity”, Nature 256(5517):495-497 (1975)

Kumar (1998)

Laemmli, “Cleavage of structural proteins during the assembly of the head of bacteriophage T4”, Nature 227(259):680-685 (1970)

Loeser, “Integrin-mediated attachment of articular chondrocytes to extracellular matrix proteins”, Arthritis Rheum. 36(8):1103-1110 (1993)

Longhurst et al, “Integrin-mediated signal transduction”, Cell Mol. Life Sci. 54(6):514-526 (1998).

Liu et al, “Chimeric mouse-human IgG1 antibody that can mediate lysis of cancer cells”, Proc. Nat. Acad. Sci USA 84(10):3439-3443 (1987)

McKee, Glimcher et al, (ID:638), “High-resolution immunolocalization of osteopontin and osteocalcin in bone and cartilage during endochondral ossification in the chicken tibia”, Anat. Rec. 234(4):479-492 (1992)

Morrison et al, “Chimeric human antibody molecules: mouse antigen-binding domains with human constant region domains”, Proc. Nat. Acad. Sci. USA 81(21):6851-6855 (1984)

Mould et al, “Defining the topology of integrin α₅β₁-fibronectin interactions using inhibitory anti-α5 and anti-β1 monoclonal antibodies. Evidence that the synergy sequence of fibronectin is recognized by the amino-terminal repeats of the α5 subunit”, J. Biol. Chem. 272(28)17283-17292 (1997)

Moule et al (1996)

Neuberger et al, “A hapten-specific chimaeric IgE antibody with human physiological effector function”, Nature 314(6008):268-270 (1985)

Nissim et al, “Antibody fragments from a ‘single pot’ phage display library as immunochemical reagents”, EMBO J. 13(3):692-698 (1994)

Osbourn et al, “Generation of a panel of related human scFv antibodies with high affinities for human CEA”, Immunotechnology 2(3):181-196 (1996)

Ostergaard et al (1998)

Palmer et al, “Selection of antibodies to cell surface determinants on mouse thymic epithelial cells using a phage display library”, Immunology 91(3):473-478 (1997)

Patel et al (1997)

Pereira et al, “A model system for detection and isolation of a tumor cell surface antigen using antibody phage display”, J. Immunol. Methods 203(1):11-24 (1997a)

Pereira et al, “Combinatorial antibodies against human malignant melanoma”, Hybridoma 16(1):11-16 (1997b)

Pfistermueller et al, “Preferential recognition of the very low-density lipoprotein receptor ligand binding site by antibodies from phage display libraries”, FEBS Lett. 396(1):14-20 (1996)

Retta et al, “β1-integrin cytoplasmic subdomains involved in dominant negative function”, Mol. Biol. Cell 9(4):715-731 (1998)

Sahagan et al, “A genetically engineered murine/human chimeric antibody retains specificity for human tumor-associated antigen”, J. Immunol. 137(3):1066-1074 (1986)

Sambrook et al, Molecular Cloning: A Laboratory Manual, 2nd. Ed., Cold Spring Harbor Laboratory Press (Cold Spring Harbor, 1989)

Sarkissian et al, “Integrin engagement regulates proliferation and collagenase expression of rheumatoid synovial fibroblasts”, J. Immunol. 162(3):1772-9 (1999)

Sawyer et al, “Methodology for selection of human antibodies to membrane proteins from a phage-display library”, J. Immunol. Methods 204(2):193-203 (1997)

Schulz et al, Principles of Protein Structure, Springer-Verlag (New York, 1978)

Shen et al, “Antibodies to different members of the β1 (CD29) integrins induce homotypic and heterotypic cellular aggregation”, Cell Immunol. 138(1):216-228 (1991)

Siegel et al, “Isolation of cell surface-specific human monoclonal antibodies using phage display and magnetically-activated cell sorting: applications in immunohematology”, J. Immunol. Methods 206(1-2):73-85 (1997)

Storgard et al, “Decreased angiogenesis and arthritic disease in rabbits treated with an αᵥβ₃ antagonist, J. Clin. Invest. 103(1):47-54 (1999)

Sun et al, “, Proc. Nat. Acad. Sci. USA 84:214-218 (1987)

Tomatis et al (1999) “The muscle-specific laminin receptor integrin negatively regulates α₅β₁ alpha5 beta1 fibronectin receptor function”, Exp. Cell Res. 1;246(2):421-32 (1999)

Trusolino et al, “Growth factor-dependent activation of alphavbeta3 integrin in normal epithelial cells: implications for tumor invasion”, J. Cell Biology 142(4):1145-1156 (1998)

Tsujii et al, “Expression of COX-1 and COX-2 and gastrointestinal diseases”, Nippon Naika Gakkai Zasshi 10;87(10):2114-21 (1998)

Vogel et al (1997)

Vuori et al, “Association of insulin receptor substrate-1 with integrins,” Science 266(5190):1576-1568 (1994).

Wahl et al, “Improved radioimaging and tumor localization with monoclonal F(ab′)2”, J. Nucl. Med. 24(4):316-325 (1983).

Watters et al, “An optimized method for cell-based phage display panning” Immunotechnology 3(1):21-29 (1997).

Weber et al, Science 271:

Weerasinghe et al, “A role for the αᵥβ₃ integrin in the transmigration of monocytes”, J. Cell Biol. 142(2):595-607 (1998)

Yonezawa et al, “VLA-5-mediated interaction with fibronectin induces cytokine production by human chondrocytes”, Biochem. Biophys. Res. Commun. 219(1):261-265 (1996).

Young et al, “cDNA cloning, mRNA distribution and heterogeneity, chromosomal location, and RFLP analysis of human osteopontin (OPN)”, Genomics 7(4):491-502 (1990).

Zheng et al, “Blocking ligand occupancy of the αᵥβ₃ integrin inhibits insulin-like growth factor I signaling in vascular smooth muscle cells”, Proc. Natl. Acad. Sci. USA 95(19):11217-11222 (1998).

What is claimed is:

1. A method for treating osteoarthritis by regulating inflammatory mediators comprising administering to a patient in need thereof an effective amount of an agent which affects at least one integrin which regulates production of inflammatory mediators.

2. The method according to claim 1, wherein the integrin is αᵥβ₃.

3. The method according to claim 2, wherein the agent binds to sequences of αᵥβ₃ to which at least one of osteopontin and LM609 bind.

4. The method according to claim 2, wherein said agent has sequences that bind to the integrin.

5. The method according to claim 1, wherein the agent is a monoclonal antibody.

6. The method according to claim 5, wherein the monoclonal antibody is LM609.

7. The method according to claim 1, wherein the agent is a compound which mimics the activity of LM609.

8. The method according to claim 1, wherein the agent is administered along with a compound selected from the group consisting of non-steroidal anti-inflammatory compounds, COX-1 inhibitors, COX-2 inhibitors, immunosuppressants, methotrexate, TNFα, and disease modifying drugs.

9. The method according to claim 1, wherein the agent is administered by gene therapy.

10. The method according to claim 1, wherein the inflammatory mediators are selected from the group consisting of IL-Iβ, IL-6, IL-8, nitric oxide, PGE₂, and MMPs.

11. The method according to claim 1, wherein the patient is suffering from a condition or disease selected from the group consisting of multiple sclerosis, type I diabetes, giant cell arthritis, systemic lupus erythematosus, Sjögren's Syndrome, rheumatoid arthritis, osteoarthritis, atherosclerosis, ischemia, arthrosclerosis, HIV infection and AIDS, bacterial infection, respiratory distress syndrome, smoking, coal mine pneumonoconiosis, alcoholic cirrhosis, cuprophane hemodialysis, cardiopulmonary bypass, chronic hepatitis B, thermal injury, reticulchistocytosis, sarcoidosis, tuberculosis, obstructive jaundice, Paget's Disease, osteomalacia, Kawasaki's Disease, inflammatory bowel disease, schistosoma infection, periodontal disease, pancreatitis, renal dysfunction, Alzheimer's Disease, atopic dermatitis, respiratory viral infection, scleroderma, cerebral malaria, uveitis, inflammatory skin diseases, chronic prostatitis, osteoporosis, hepatic fibrosis, host versus graft disease, proliferation of chronic myelogenous leukemia cells, organ transplantation, local inflammatory lesions, acute phase response, vasculitis, and septic shock.

12. At least one sequence of an αᵥβ₃ epitope to which LM609 binds.

13. At least one sequence of osteopontin that binds to an αᵥβ₃ integrin.

14. A method for preventing or treating osteoarthritis involving inflammation comprising administering to a patient in need thereof an effective amount of an agent which affects at least one integrin which regulates production of inflammatory mediators.

15. The method according to claim 14, wherein the integrin is αᵥβ₃.

16. The method according to claim 15, wherein the agent binds to sequences of αᵥβ₃ to which at least one of osteopontin and LM609 bind.

17. The method according to claim 15, wherein said agent has sequences that bind to the integrin.

18. The method according to claim 14, wherein the agent is a monoclonal antibody.

19. The method according to claim 18, wherein the monoclonal antibody is LM609.

20. The method according to claim 14, wherein the agent is a compound which mimics the activity of LM609.

21. The method according to claim 14, wherein the agent is administered along with a compound selected from the group consisting of non-steroidal anti-inflammatory compounds, COX-1 inhibitors, COX-2 inhibitors, immunosuppressants, methotrexate, TNFα, and disease modifying drugs.

22. The method according to claim 14, wherein the agent is administered by gene therapy.

23. A method for inhibiting the interaction of fibronectin with integrin comprising administering an effective amount of JCS5 antibody to bind the binding site of fibronectin.

24. A method according to claim 23 further comprsing adding an antisense to the α₅β₁ receptor of integrin.

25. The method according to claim 23 further comprising adding a cyclic RGD peptide.

26. The method according to claim 14, wherein the inflammatory mediators are selected from the group consisting of IL-1β, IL-6, IL-8, nitric oxide, PGE2, and MMPs.

27. The method according to claim 14, wherein the patient is suffering from a condition or disease selected from the consisting of multiple sclerosis, type I diabetes, giant cell arthritis, systemic lupus erythematosus, Sjögren's Syndrome, rheumatoid arthritis, osteoarthritis, atherosclerosis, ischemia, arthrosclerosis, HIV infection and AIDS, bacterial infection, respiratory distress syndrome, smoking, coal mine pneumonoconiosis, alcoholic cirrhosis, cuprophane hemodialysis, cardiopulmonary bypass, chronic hepatitis B, thermal injury, reticulohistocytosis, sarcoidosis, tuberculosis, obstructive jaundice, Paget's Disease, osteomalacia, Kawasaki's Disease, inflammatory bowel disease, schistosoma infection, periodontal disease, pancreatitis, renal dysfunction, Alzheimer's Disease, atopic dermatitis, respiratory viral infection, scleroderma, cerebral malaria, uveitis, inflammatory skin diseases, chronic prostatitis, osteoporosis, hepatic fibrosis, host versus graft disease, proliferation of chronic myelogenous leukemia cells, organ transplantation, local inflammatory lesions, acute phase response, vasculitis, and septic shock.

28. A method of inhibiting inflammatory mediators and IL-1 mediated functions comprising treating a patient in need thereof by gene therapy to express an effective amount of an agent which affects at least one integrin which regulates production of inflammatory mediators.

29. A method for identifying anti-inflammatory compounds or compounds which act as mediators for inflammation comprising contacting a compound with an effective amount of an agent which affects at least one integrin which regulates production of inflammatory mediators and detecting reaction of said agent with said compound.

30. A method for confirming that a compound administered acts on inflammatory mediators comprising contacting a compound with an effective amount of an agent which affects at least one integrin which regulates production of inflammatory mediators and detecting reaction of said agent with said compound.


SEQUENCE LISTING (6 sequences)

SEQ ID NO: 1 - DNA, 22 bp, Artificial Sequence
  synthetic
gcgcctggtcacccagggctgc

SEQ ID NO: 2 - DNA, 26 bp, Artificial Sequence
  synthetic
ggatcctcccccgctcctggaagatc

SEQ ID NO: 3 - DNA, 20 bp, Artificial Sequence
  synthetic
gaagagctgcatccaacacc

SEQ ID NO: 4 - DNA, 22 bp, Artificial Sequence
  synthetic
atgcagaacacccaccttctcg

SEQ ID NO: 5 - DNA, 27 bp, Artificial Sequence
  synthetic
cgggatccatgttgcgcttgtacgtgt

SEQ ID NO: 6 - DNA, 30 bp, Artificial Sequence
  synthetic
taaagcggccgctcacttgggatagaattg